(19) United States
(12) Patent Application Publication
(10) Pub. No.: US 2016/0289188 A1
(43) Pub. Date: Oct. 6, 2016

(54) COMPOSITIONS AND METHODS FOR THE TREATMENT OF VIRAL DISEASES WITH PDE4 MODULATORS

(71) Applicant: CELGENE CORPORATION, Summit, NJ (US)

(72) Inventors: Jerome B. Zeldis, Princeton, NJ (US); Vikram Khetani, Short Hills, NJ (US)

(21) Appl. No.: 15/034,854

(22) PCT Filed: Nov. 5, 2014
(86) PCT No.: PCT/US14/64047

§ 371 Date: May 5, 2016

Related U.S. Application Data
(60) Provisional application No. 61/900,489, filed on Nov. 6, 2013.

(51) Int. Cl. C07D 209/46; A61K 31/7056; A61K 31/4035

(52) U.S. Cl. CPC C07D 209/46; A61K 31/4035; A61K 31/7056

(57) ABSTRACT

Methods of treating and/or preventing viral diseases and disorders, such as those ameliorated by inhibition of PDE-4, are disclosed.

### 1 FIELD OF THE INVENTION

The present invention relates to treating or preventing a virus induced disease or condition comprising administering to a patient in need thereof an effective amount of a PDE4 modulator. In certain embodiments, the PDE4 modulator is selected from 3-(3,4-dimethoxyphenyl)-3-(1-oxoindolin-2-yl)propionamide (Compound A) or a pharmaceutically acceptable salt, solvate, hydrate, clathrate, stereoisomer, or prodrug thereof; and N-(2-(1-(3-ethoxy-4-methoxyphenyl)-2-(methylsulfonyl)ethyl)-3-oxoisoindolin-4-yl)cyclopropanecarboxamide (Compound B) or a pharmaceutically acceptable salt, solvate, hydrate, clathrate, stereoisomer, or prodrug thereof. Such viral diseases include, but are not limited to Argentine Hemorrhagic Fever, Lassa Fever, Influenza, and other viruses whose pathophysiology involves the production of a cytokine storm.

### 2 BACKGROUND OF THE INVENTION

3-(3,4-dimethoxyphenyl)-3-(1-oxoindolin-2-yl)propionamide (Compound A) is a compound that inhibits phosphodiesterase type IV (PDE4). Compound A and methods for its synthesis are described, e.g., in U.S. Pat. No. 5,698,579, the disclosure of which is hereby incorporated by reference in its entirety.

Cyclopropanecarboxylic acid {2-[(1S)-1-(3-ethoxy-4-methoxy-phenyl)-2-methanesulfonyl-ethyl]-3-oxo-2,3-dihydro-1H-isoindol-4-yl}-amide (Compound B) is a compound that inhibits phosphodiesterase type IV (PDE4) enzyme. Compound B and methods for its synthesis are described, e.g., in U.S. Pat. No. 6,667,316, the disclosure of which is hereby incorporated by reference in its entirety.

### 3 SUMMARY OF THE INVENTION

In certain embodiments, the invention relates to methods for treating or preventing a disease ameliorated by modulation of PDE4, comprising administering to a patient in need thereof an effective amount of a PDE4 modulator as disclosed herein. In certain embodiments the PDE modulator is Compound A, Compound B, or a pharmaceutically acceptable salt, solvate, hydrate, clathrate, stereoisomer, or prodrug thereof. In certain such embodiments, the invention relates to methods for treating or preventing a virus induced disease or condition ameliorated by modulation of PDE4, comprising administering to a patient in need thereof an effective amount of Compound A, Compound B, or a pharmaceutically acceptable salt, solvate, hydrate, clathrate, stereoisomer, or prodrug thereof.

In certain embodiments, the invention relates to methods for treating or preventing a disease ameliorated by inhibition of PDE4, comprising administering to a patient in need thereof an effective amount of a PDE4 inhibitor as disclosed herein. In certain embodiments the PDE inhibitor is Compound A, Compound B, or a pharmaceutically acceptable salt, solvate, hydrate, clathrate, stereoisomer, or prodrug thereof. In certain such embodiments, the invention relates to methods for treating or preventing a virus induced disease or condition ameliorated by inhibition of PDE4, comprising administering to a patient in need thereof an effective amount of Compound A, Compound B, or a pharmaceutically acceptable salt, solvate, hydrate, clathrate, stereoisomer, or prodrug thereof. In certain embodiments, the invention relates to methods for treating or preventing a virus induced disease or condition whose pathophysiology involves the production of a cytokine storm, comprising administering to a patient in need thereof an effective amount of a PDE4 inhibitor as disclosed herein. In certain embodiments the PDE inhibitor is Compound A, Compound B, or a pharmaceutically acceptable salt, solvate, hydrate, clathrate, stereoisomer, or prodrug thereof.

In certain embodiments, the invention relates to methods for down modulating monocyte and macrophage driven cytokine storms, comprising administering to a patient in need thereof an effective amount of a PDE4 inhibitor as disclosed herein. In certain embodiments the PDE inhibitor is Compound A, Compound B, or a pharmaceutically acceptable salt, solvate, hydrate, clathrate, stereoisomer, or prodrug thereof. In certain embodiments, the invention relates to methods for treating or preventing a virus-induced monocyte and macrophage cytokine storm, comprising administering to a patient in need thereof an effective amount of a PDE4 inhibitor as disclosed herein. In certain embodiments the PDE inhibitor is Compound A, Compound B, or a pharmaceutically acceptable salt, solvate, hydrate, clathrate, stereoisomer, or prodrug thereof.

### 4 BRIEF DESCRIPTION OF THE FIGURES

FIG. 1 shows the percent survival of animals treated with Compound A, Compound B, Ribavirin, or placebo after infection with Junin virus.

FIG. 2 shows the average temperature in animals treated with Compound A or Compound B in the absence of exposure to Junin virus (Groups 3 and 4).

FIG. 3 shows the average weight of animals treated with Compound A or Compound B in the absence of exposure to Junin virus (Groups 3 and 4).

FIG. 4 shows the average temperature in animals treated with Compound A, Compound B, Ribavirin, or placebo after infection with Junin virus (Groups 1, 2, 5, and 6).

FIG. 5 shows the average weight in animals treated with Compound A, Compound B, Ribavirin, or placebo after infection with Junin virus (Groups 1, 2, 5, and 6).

FIG. 6 shows the percent survival of animals treated with Compound A, Compound B, Ribavirin, or placebo, either once or twice daily after infection with Junin virus.

FIG. 7 shows the average temperature of animals treated with Compound A, Compound B, Ribavirin, or placebo, either once or twice daily after infection with Junin virus.

FIG. 8 shows the average weight of animals treated with Compound A, Compound B, Ribavirin, or placebo, either once or twice daily after infection with Junin virus.

FIG. 9 shows the percent survival of animals treated with Compound A, Compound B, Ribavirin, or placebo, either once or twice daily after infection with Lassa virus.

FIG. 10 shows the average weight of animals treated with Compound A, Compound B, Ribavirin, or placebo, either once or twice daily after infection with Lassa virus.

FIG. 11 shows the average weight of animals treated with Compound A, Compound B, Ribavirin, or placebo, either once or twice daily after infection with Lassa virus.

### 5 DETAILED DESCRIPTION

### 5.1 Definitions

As used herein, the term “Compound A” refers to 3-(3,4-dimethoxy-phenyl)-3-(1-oxo-1,3-dihydro-isoindol-2-yl)-propionamide which has the following structure

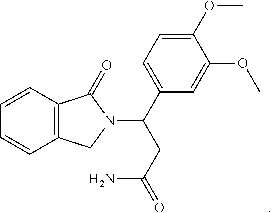 [SMILES: COc1ccc(C(CC(N)=O)N2Cc3ccccc3C2=O)cc1OC ; "."]

or any stereoisomer or any mixture stereoisomers thereof. Compound A also refers to any crystal structure or polymorph of 3-(3,4-dimethoxy-phenyl)-3-(1-oxo-1,3-dihydro-isoindol-2-yl)-propionamide.

As used herein, the term “Compound B” refers to N-(2-(1-(3-ethoxy-4-methoxyphenyl)-2-(methylsulfonyl)ethyl)-3-oxoisoindolin-4-yl)cyclopropanecarboxamide which has the following structure

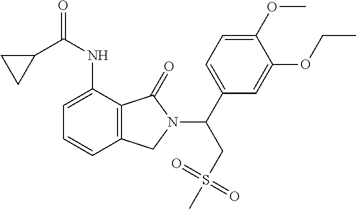 [SMILES: CCOc1cc(C(CS(C)(=O)=O)N2Cc3cccc(NC(=O)C4CC4)c3C2=O)ccc1OC]

or any stereoisomer or any mixture stereoisomers thereof, such as (S)—N-(2-(1-(3-ethoxy-4-methoxyphenyl)-2-(methylsulfonyl)ethyl)-3-oxoisoindolin-4-yl)cyclopropanecarboxamide. Compound B also refers to any crystal structure or polymorph of N-(2-(1-(3-ethoxy-4-methoxyphenyl)-2-(methylsulfonyl)ethyl)-3-oxoisoindolin-4-yl)cyclopropanecarboxamide.

As used herein, the term “patient” or “subject” means an animal (e.g., cow, horse, sheep, pig, chicken, turkey, quail, cat, dog, mouse, rat, rabbit, guinea pig, etc.), preferably a mammal such as a non-primate or a primate (e.g., monkey and human), most preferably a human. In certain embodiments, the patient is a fetus, embryo, infant, child, adolescent or adult.

As used herein, an “effective amount” refers to that amount of Compound A, Compound B, or a pharmaceutically acceptable salt, solvate or hydrate thereof sufficient to provide a therapeutic benefit in the treatment of the disease or to delay or minimize symptoms associated with the disease. Certain preferred effective amounts are described herein.

As used herein, the terms “prevent”, “preventing” and “prevention” are art-recognized, and when used in relation to a condition, such as a local recurrence (e.g., pain), a disease such as cancer, a syndrome complex such as heart failure or any other medical condition, is well understood in the art, and includes administration of a compound, such as Compound A, Compound B, or a pharmaceutically acceptable salt, solvate or hydrate thereof, which reduces the frequency of, or delays the onset of, symptoms of a medical condition in a subject relative to a subject which does not receive the composition.

As used herein, the terms “treat”, “treating” and “treatment” refer to the reversing, reducing, or arresting the symptoms, clinical signs, and underlying pathology of a condition in manner to improve or stabilize a subject's condition. The terms “treat” and “treatment” also refer to the eradication or amelioration of the disease or symptoms associated with the disease. In certain embodiments, such terms refer to minimizing the spread or worsening of the disease resulting from the administration of Compound A, Compound B, or a pharmaceutically acceptable salt, solvate or hydrate thereof to a patient with such a disease.

As used herein, the term “pharmaceutically acceptable salt” refers to a salt prepared from pharmaceutically acceptable non-toxic acids or bases including inorganic acids and bases and organic acids and bases. Suitable pharmaceutically acceptable base addition salts include, but are not limited to, sodium, lithium, potassium, calcium, magnesium, zinc, bismuth, ammonium, lysine, tromethaminie, and meglumine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Other examples of salts are well known in the art, see, e.g., Remington's Pharmaceutical Sciences, 22nd ed., Pharmaceutical Press, (2012).

As used herein, the term “hydrate” means a compound as disclosed herein, such as Compound A or Compound B, or a pharmaceutically acceptable salt thereof, that further includes a stoichiometric or non-stoichiometric amount of water bound by non-covalent intermolecular forces.

As used herein, the term “solvate” means a compound as disclosed herein, such as Compound A, Compound B or a pharmaceutically acceptable salt thereof, that further includes a stoichiometric or non-stoichiometric amount of a solvent, other than water, bound by non-covalent intermolecular forces.

As used herein and unless otherwise indicated, the term “PDE4 modulators” encompasses small molecule drugs, e.g., small organic molecules which are not peptides, proteins, nucleic acids, oligosaccharides or other macromolecules. In certain embodiments, the compounds inhibit TNF-α production. Compounds may also have an inhibitory effect on LPS induced IL1β and IL12. In some embodiments, the compounds are potent PDE4 inhibitors. Without being bound by theory, in certain embodiments, PDE4 inhibitors as disclosed herein, such as Compound A, Compound B, or a pharmaceutically acceptable salt, solvate or hydrate thereof down modulate monocyte driven cytokine storms. Thus, any virus-induced monocyte and macrophage cytokine storm may be ameliorated by a PDE4 inhibitor.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO2, —ONO, or —ONO2 moieties. Prodrugs can typically be prepared using well-known methods, such as those described in Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, and unless otherwise specified, the term “stereoisomer” encompasses all enantiomerically/stereomerically pure and enantiomerically/stereomerically enriched compounds provided herein.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer. In certain cases, a compound provided herein is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds provided herein (e.g., R/S=30/70, 35/65, 40/60, 45/55, 55/45, 60/40, 65/35 and 70/30).

As used herein, and unless otherwise specified, the term “therapeutically effective amount” of a compound is an amount sufficient to provide a therapeutic benefit in the treatment of a disease or condition, or to delay or minimize one or more symptoms associated with the disease or condition. A therapeutically effective amount of a compound means an amount of therapeutic agent, alone or in combination with other therapies, which provides a therapeutic benefit in the treatment of the disease or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, the term “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

### 5.2 PDE4-Modulators

Compounds provided herein include racemic, stereomerically pure and stereomerically enriched PDE4 modulators, stereomerically and enantiomerically pure compounds that have selective cytokine inhibitory activities, and pharmaceutically acceptable salts, solvates, hydrates, stereoisomers, clathrates, and prodrugs thereof. In certain embodiments, compounds are known PDE4 modulators of Celgene Corporation, NJ.

Examples of PDE4 modulators include, but are not limited to, the cyclic imides disclosed in U.S. Pat. Nos. 5,605,914 and 5,463,063; the cycloalkyl amides and cycloalkyl nitriles of U.S. Pat. Nos. 5,728,844, 5,728,845, 5,968,945, 6,180,644 and 6,518,281; the aryl amides (for example, an embodiment being N-benzoyl-3-amino-3-(3′,4′-dimethoxyphenyl)-propanamide) of U.S. Pat. Nos. 5,801,195, 5,736,570, 6,046,221 and 6,284,780; the imide/amide ethers and alcohols (for example, 3-phthalimido-3-(3′,4′-dimethoxyphenyl)propan-1-ol) disclosed in U.S. Pat. No. 5,703,098; the succinimides and maleimides (for example methyl 3-(3′,4′,5′6′-petrahydrophthalimdo)-3-(3″,4″-dimethoxyphenyl)propionate) disclosed in U.S. Pat. No. 5,658,940; imido and amido substituted alkanohydroxamic acids disclosed in U.S. Pat. No. 6,214,857 and WO 99/06041; substituted phenethylsulfones disclosed in U.S. Pat. Nos. 6,011,050 and 6,020,358; fluoroalkoxy-substituted 1,3-dihydro-isoindolyl compounds disclosed in U.S. Pat. No. 7,173,058; substituted imides (for example, 2-phthalimido-3-(3′,4′-dimethoxyphenyl) propane) disclosed in U.S. Pat. No. 6,429,221; substituted 1,3,4-oxadiazoles (for example, 2-[1-(3-cyclopentyloxy-4-methoxyphenyl)-2-(1,3,4-oxadiazole-2-yl)ethyl]-5-methylisoindoline-1,3-dione) disclosed in U.S. Pat. No. 6,326,388; cyano and carboxy derivatives of substituted styrenes (for example, 3,3-bis-(3,4-dimethoxyphenyl) acrylonitrile) disclosed in U.S. Pat. Nos. 5,929,117, 6,130,226, 6,262,101 and 6,479,554; isoindoline-1-one and isoindoline-1,3-dione substituted in the 2-position with an α-(3,4-disubstituted phenyl)alkyl group and in the 4- and/or 5-position with a nitrogen-containing group disclosed in WO 01/34606 and U.S. Pat. No. 6,667,316, for example, cyclopropyl-N-{2-[1-(3-ethoxy-4-methoxyphenyl)-2-(methylsulfonyl)ethyl]-3-oxoisoindolin-4-yl}carboxamide, cyclopropyl-N-{2-[1(S)-(3-ethoxy-4-methoxyphenyl)-2-(methylsulfonyl)ethyl]-3-oxoisoindolin-4-yl}carboxamide, and cyclopropyl-N-{2-[1(R)-(3-ethoxy-4-methoxyphenyl)-2-(methylsulfonyl)ethyl]-3-oxoisoindolin-4-yl}carboxamide; and imido and amido substituted acylhydroxamic acids (for example, (3-(1,3-dioxoisoindoline-2-yl)-3-(3-ethoxy-4-methoxyphenyl) propanoylamino) propanoate disclosed in WO 01/45702 and U.S. Pat. No. 6,699,899. Other PDE4 modulators include diphenylethylene compounds disclosed in U.S. Pat. No. 7,312,241, the contents of which are incorporated by reference herein in their entirety. Other PDE4 modulators include isoindoline compounds disclosed in U.S. patent publication no. 2006/0025457A1, published Feb. 2, 2006 and U.S. Pat. No. 7,244,759. Other specific PDE4 modulators include 2-[1-(3-ethoxy-4-methoxyphenyl)-2-methylsulfonylethyl]-4-acetylaminoisoindoline-1,3-dione, and stereoisomers thereof (+)-2-[1-(3-ethoxy-4-methoxyphenyl)-2-methylsulfonylethyl]-4-acetylaminoisoindoline-1,3-dione was disclosed in WO 03/080049. The entireties of each of the patents and patent applications identified herein are incorporated herein by reference.

Additional PDE4 modulators belong to a family of synthesized chemical compounds of which typical embodiments include 3-(1,3-dioxobenzo-[f]isoindol-2-yl)-3-(3-cyclopentyloxy-4-methoxyphenyl)propionamide and 3-(1,3-dioxo-4-azaisoindol-2-yl)-3-(3,4-dimethoxyphenyl)-propionamide.

Other PDE4 modulators belong to a class of non-polypeptide cyclic amides disclosed in U.S. Pat. Nos. 5,698,579, 5,877,200, 6,075,041 and 6,200,987, and WO 95/01348, each of which is incorporated herein by reference. Representative cyclic amides include compounds of the formula:

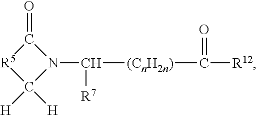

or a pharmaceutically acceptable salt, solvate, hydrate, clathrate, stereoisomer, or prodrug thereof, wherein:
n has a value of 1, 2, or 3;
R⁵ is o-phenylene, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkyl of 1 to 10 carbon atoms, or halo;
R⁷ is (i) phenyl or phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo; (ii) benzyl unsubstituted or substituted with 1 to 3 substituents selected from the group consisting of nitro, cyano, trifluoromethyl, carbothoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo; (iii) naphthyl; or (iv) benzyloxy;
R¹² is —OH, alkoxy of 1 to 12 carbon atoms, or

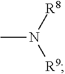

R⁸ is hydrogen or alkyl of 1 to 10 carbon atoms; and
R⁹ is hydrogen, alkyl of 1 to 10 carbon atoms, —COR¹⁰, or —SO₂R¹⁰,
R¹⁰ is hydrogen, alkyl of 1 to 10 carbon atoms, or phenyl.

In some embodiments, the PDE4 modulator selected from:
3-phenyl-2-(1-oxoisoindolin-2-yl)propionic acid;
3-phenyl-2-(1-oxoisoindolin-2-yl)propionamide;
3-phenyl-3-(1-oxoisoindolin-2-yl)propionic acid;
3-phenyl-3-(1-oxoisoindolin-2-yl)propionamide;
3-(4-methoxyphenyl)-3-(1-oxisoindolin-yl)propionic acid;
3-(4-methoxyphenyl)-3-(1-oxisoindolin-yl)propionamide;
3-(3,4-dimethoxyphenyl)-3-(1-oxisoindolin-2-yl)propionic acid;
3-(3,4-dimethoxy-phenyl)-3-(1-oxo-1,3-dihydroisoindol-2-yl)propionamide;
3-(3,4-dimethoxyphenyl)-3-(1-oxisoindolin-2-yl)propionamide;
3-(3,4-diethoxyphenyl)-3-(1-oxoisoindolin-yl)propionic acid;
methyl 3-(1-oxoisoindolin-2-yl)-3-(3-ethoxy-4-methoxyphenyl)propionate;
3-(1-oxoisoindolin-2-yl)-3-(3-ethoxy-4-methoxyphenyl)propionic acid;
3-(1-oxoisoindolin-2-yl)-3-(3-propoxy-4-methoxyphenyl)propionic acid;
3-(1-oxoisoindolin-2-yl)-3-(3-butoxy-4-methoxyphenyl)propionic acid;
3-(1-oxoisoindolin-2-yl)-3-(3-propoxy-4-methoxyphenyl)propionamide;
3-(1-oxoisoindolin-2-yl)-3-(3-butoxy-4-methoxyphenyl)propionamide;
methyl 3-(1-oxoisoindolin-2-yl)-3-(3-butoxy-4-methoxyphenyl)propionate; and
methyl 3-(1-oxoisoindolin-2-yl)-3-(3-propoxy-4-methoxyphenyl)propionate
   or a pharmaceutically acceptable salt, solvate, hydrate, clathrate, stereoisomer, or prodrug thereof.

Other representative cyclic amides include compounds of the formula:

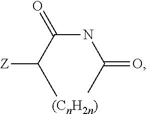

or a pharmaceutically acceptable salt, solvate, hydrate, clathrate, stereoisomer, or prodrug thereof,
wherein

### Z is:

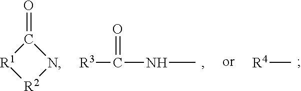

R¹ is the divalent residue of (i) 3,4-pyridine, (ii) pyrrolidine, (iii) imidizole, (iv) naphthalene, (v) thiophene, or (vi) a straight or branched alkane of 2 to 6 carbon atoms, unsubstituted or substituted with phenyl or phenyl substituted with nitro, cyano, trifluoromethyl, carbethoxy, carbomethoxy, carbopropoxy, acetyl, carbamyl, acetoxy, carboxy, hydroxy, amino, alkyl of 1 to 10 carbon atoms, alkoxy of 1 to 10 carbon atoms, or halo, wherein the divalent bonds of said residue are on vicinal ring carbon atoms;
R² is —CO— or —SO₂—;
R³ is (i) phenyl substituted with 1 to 3 substituents each selected independently from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (ii) pyridyl; (iii) pyrrolyl; (iv) imidazolyl; (iv) naphthyl; (vi) thienyl; (vii) quinolyl; (viii) furyl; or (ix) indolyl;
R⁴ is alanyl, arginyl, glycyl, phenylglycyl, histidyl, leucyl, isoleucyl, lysyl, methionyl, prolyl, sarcosyl, seryl, homoseryl, threonyl, thyronyl, tyrosyl, valyl, benzimidol-2-yl, benzoxazol-2-yl, phenylsulfonyl, methylphenylsulfonyl, or phenylcarbamoyl; and n has a value of 1, 2, or 3.

Other representative cyclic amides include compounds of the formula:

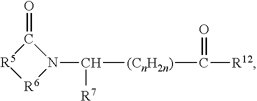

or a pharmaceutically acceptable salt, solvate, hydrate, clathrate, stereoisomer, or prodrug thereof, wherein:
R⁵ is (i) o-phenylene, unsubstituted or substituted with 1 to 4 substituents each selected independently from nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, or halo; or (ii) the divalent residue of pyridine, pyrrolidine, imidizole, naphthalene, or thiophene, wherein the divalent bonds are on vicinal ring carbon atoms;
R⁶ is —CO—, —CH₂—, or —SO₂—;
R⁷ is (i) hydrogen if R⁶ is —SO₂—; (ii) straight, branched, or cyclic alkyl of 1 to 12 carbon atoms; (iii) pyridyl; (iv) phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (v) alkyl of 1 to 10 carbon atoms; (vi) benzyl unsubstituted or substituted with 1 to 3 substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (vii) naphthyl; (viii) benzyloxy; or (ix) imidazol-4-yl methyl;
R¹² is —OH, alkoxy of 1 to 12 carbon atoms, or

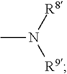

n has a value of 0, 1, 2, or 3;
R^{8′} is hydrogen or alkyl of 1 to 10 carbon atoms; and
R^{9′} is hydrogen, alkyl of 1 to 10 carbon atoms, —COR¹⁰, or —SO₂R¹⁰ in which R¹⁰ is hydrogen, alkyl of 1 to 10 carbon atoms, or phenyl.

Other representative imides include compounds of the formula:

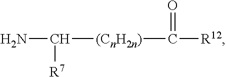

or a pharmaceutically acceptable salt, solvate, hydrate, clathrate, stereoisomer, or prodrug thereof, wherein:
R⁷ is (i) straight, branched, or cyclic alkyl of 1 to 12 carbon atoms; (ii) pyridyl; (iii) phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, or halo; (iv) benzyl unsubstituted or substituted with one to three substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 4 carbon atoms, or halo; (v) naphthyl; (vi) benzyloxy; or (vii) imidazol-4-ylmethyl;
R¹² is —OH, alkoxy of 1 to 12 carbon atoms, —O—CH₂-pyridyl, —O-benzyl or

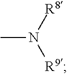

where n has a value of 0, 1, 2, or 3;
R^{8′} is hydrogen or alkyl of 1 to 10 carbon atoms;
R^{9′} is hydrogen, alkyl of 1 to 10 carbon atoms, —CH₂-pyridyl, benzyl, —COR¹⁰, or —SO₂R¹⁰; and
R¹⁰ is hydrogen, alkyl of 1 to 4 carbon atoms, or phenyl.

Other PDE4 modulators include the imido and amido substituted alkanohydroxamic acids disclosed in WO 99/06041 and U.S. Pat. No. 6,214,857, each of which is incorporated herein by reference. Examples of such compounds include, but are not limited to:

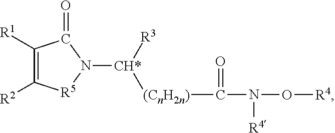

or a pharmaceutically acceptable salt, solvate, hydrate, clathrate, stereoisomer, or prodrug thereof, wherein:
each of R¹ and R², when taken independently of each other, is hydrogen, lower alkyl; or R¹ and R², when taken together with the depicted carbon atoms to which each is bound, is o-phenylene, o-naphthylene, or cyclohexene-1,2-diyl, unsubstituted or substituted with 1 to 4 substituents each selected independently from the group consisting of nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, alkoxy of 1 to 10 carbon atoms, and halo;
R³ is phenyl substituted with from one to four substituents selected from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, alkylthio of 1 to 10 carbon atoms, benzyloxy, cycloalkoxy of 3 to 6 carbon atoms, C₄-C₆-cycloalkylidenemethyl, C₃-C₁₀-alkylidenemethyl, indanyloxy, and halo;
R⁴ is hydrogen, alkyl of 1 to 6 carbon atoms, phenyl, or benzyl;
R^{4′} is hydrogen or alkyl of 1 to 6 carbon atoms;
R⁵ is —CH₂—, —CH₂—CO—, —SO₂—, —S—, or —NHCO—; and
n has a value of 0, 1, or 2.

In some embodiments, the PDE4 modulator is the following compound, or a pharmaceutically acceptable salt, solvate, hydrate, clathrate, stereoisomer, or prodrug thereof:
3-(3-ethoxy-4-methoxyphenyl)-N-hydroxy-3-(1-oxoisoindolinyl)propionamide;
3-(3-ethoxy-4-methoxyphenyl)-N-methoxy-3-(1-oxoisoindolinyl)propionamide;
N-benzyloxy-3-(3-ethoxy-4-methoxyphenyl)-3-phthalimidopropionamide;
N-benzyloxy-3-(3-ethoxy-4-methoxyphenyl)-3-(3-nitrophthalimido)propionamide;
N-benzyloxy-3-(3-ethoxy-4-methoxyphenyl)-3-(1-oxoisoindolinyl)propionamide;
3-(3-ethoxy-4-methoxyphenyl)-N-hydroxy-3-phthalimidopropionamide;
N-hydroxy-3-(3,4-dimethoxyphenyl)-3-phthalimidopropionamide;
3-(3-ethoxy-4-methoxyphenyl)-N-hydroxy-3-(3-nitrophthalimido)propionamide;
N-hydroxy-3-(3,4-dimethoxyphenyl)-3-(1-oxoisoindolinyl)propionamide;
3-(3-ethoxy-4-methoxyphenyl)-N-hydroxy-3-(4-methyl-phthalimido)propionamide;
3-(3-cyclopentyloxy-4-methoxyphenyl)-N-hydroxy-3-phthalimidopropionamide;
3-(3-ethoxy-4-methoxyphenyl)-N-hydroxy-3-(1,3-dioxo-2,3-dihydro-1H-benzo[f]isoindol-2-yl)propionamide;
N-hydroxy-3-{3-(2-propoxy)-4-methoxyphenyl}-3-phthalimidopropionamide;
3-(3-ethoxy-4-methoxyphenyl)-3-(3,6-difluorophthalimido)-N-hydroxypropionamide;
3-(4-aminophthalimido)-3-(3-ethoxy-4-methoxyphenyl)-N-hydroxypropionamide;
3-(3-aminophthalimido)-3-(3-ethoxy-4-methoxyphenyl)-N-hydroxypropionamide;
3-(3-acetoamidophthalimido)-3-(3-ethoxy-4-methoxyphenyl)-N-hydroxypropionamide;
N-hydroxy-3-(3,4-dimethoxyphenyl)-3-(1-oxoisoindolinyl)propionamide;
3-(3-cyclopentyloxy-4-methoxyphenyl)-N-hydroxy-3-(1-oxoisoindolinyl) propionamide; or
N-benzyloxy-3-(3-ethoxy-4-methoxyphenyl)-3-(3-nitrophthalimido)propionamide.

Other PDE4 modulators include the substituted phenethylsulfones substituted on the phenyl group with a oxoisoindine group. Examples of such compounds include, but are not limited to, those disclosed in U.S. Pat. No. 6,020,358, which is incorporated herein by reference, which include the following:

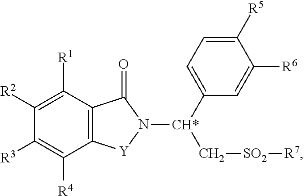

or a pharmaceutically acceptable salt, solvate, hydrate, clathrate, stereoisomer, or prodrug thereof, wherein:
the carbon atom designated * constitutes a center of chirality;
Y is C═O, CH₂, SO₂, or CH₂C═O; each of R¹, R², R³, and R⁴, independently of the others, is hydrogen, halo, alkyl of 1 to 4 carbon atoms, alkoxy of 1 to 4 carbon atoms, nitro, cyano, hydroxy, or —NR⁸R⁹; or any two of R¹, R², R³, and R⁴ on adjacent carbon atoms, together with the depicted phenylene ring are naphthylidene;
each of R⁵ and R⁶, independently of the other, is hydrogen, alkyl of 1 to 4 carbon atoms, alkoxy of 1 to 4 carbon atoms, cyano, or cycloalkoxy of up to 18 carbon atoms;
R⁷ is hydroxy, alkyl of 1 to 8 carbon atoms, phenyl, benzyl, or NR^{8′}R^{9′};
each of R⁸ and R⁹ taken independently of the other is hydrogen, alkyl of 1 to 8 carbon atoms, phenyl, or benzyl; or one of R⁸ and R⁹ is hydrogen and the other is —COR¹⁰ or —SO₂R¹⁰; or R⁸ and R⁹ taken together are tetramethylene, pentamethylene, hexamethylene, or —CH₂CH₂X¹CH₂CH₂— in which X¹ is —O—, —S— or —NH—; and
each of R^{8′} and R^{9′} taken independently of the other is hydrogen, alkyl of 1 to 8 carbon atoms, phenyl, or benzyl; or one of R^{8′} and R^{9′} is hydrogen and the other is —COR¹⁰ or —SO₂R^{10′}; or R^{8′} and R^{9′} taken together are tetramethylene, pentamethylene, hexamethylene, or —CH₂CH₂X²CH₂CH₂— in which X² is —O—, —S—, or —NH—.

It will be appreciated that while for convenience the above compounds are identified as phenethylsulfones, they include sulfonamides when R⁷ is NR^{8′}R^{9′}.

In some embodiments, the compounds are those in which Y is C═O or CH₂.

In other embodiments, the compounds are those in which each of R¹, R², R³, and R⁴ independently of the others, is hydrogen, halo, methyl, ethyl, methoxy, ethoxy, nitro, cyano, hydroxy, or —NR⁸R⁹ in which each of R⁸ and R⁹ taken independently of the other is hydrogen or methyl or one of R⁸ and R⁹ is hydrogen and the other is —COCH₃.

In other embodiments, the compounds are those in which one of R¹, R², R³, and R⁴ is —NH₂ and the remaining of R¹, R², R³, and R⁴ are hydrogen.

In other embodiments, the compounds are those in which one of R¹, R², R³, and R⁴ is —NHCOCH₃ and the remaining of R¹, R², R³, and R⁴ are hydrogen.

In other embodiments, the compounds are those in which one of R¹, R², R³, and R⁴ is —N(CH₃)₂ and the remaining of R¹, R², R³, and R⁴ are hydrogen.

In other embodiments, the compounds are those in which one of R¹, R², R³, and R⁴ is methyl and the remaining of R¹, R², R³, and R⁴ are hydrogen.

In other embodiments, the compounds are those in which one of R¹, R², R³, and R⁴ is fluoro and the remaining of R¹, R², R³, and R⁴ are hydrogen.

In other embodiments, the compounds are those in which each of R⁵ and R⁶, independently of the other, is hydrogen, methyl, ethyl, propyl, methoxy, ethoxy, propoxy, cyclopentoxy, or cyclohexoxy.

In other embodiments, the compounds are those in which R⁵ is methoxy and R⁶ is monocycloalkoxy, polycycloalkoxy, and benzocycloalkoxy.

In other embodiments, the compounds are those in which R⁵ is methoxy and R⁶ is ethoxy.

In other embodiments, the compounds are those in which R⁷ is hydroxy, methyl, ethyl, phenyl, benzyl, or NR^{8′}R^{9′} in which each of R^{8′} and R^{9′} taken independently of the other is hydrogen or methyl.

In other embodiments, the compounds are those in which R⁷ is methyl, ethyl, phenyl, benzyl or NR^{8′}R^{9′} in which each of R^{8′} and R^{9′} taken independently of the other is hydrogen or methyl.

In other embodiments, the compounds are those in which R⁷ is methyl.

In other embodiments, the compounds are those in which R⁷ is NR⁸R^{9′} in which each of R^{8′} and R^{9′} taken independently of the other is hydrogen or methyl.

Other PDE4 modulators include fluoroalkoxy-substituted 1,3-dihydro-isoindolyl compounds disclosed in U.S. Pat. No. 7,173,058, which is incorporated herein by reference. Representative compounds include those of formula:

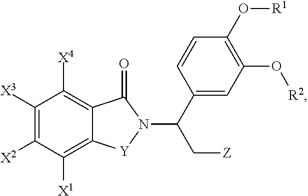

or a pharmaceutically acceptable salt, solvate, hydrate, clathrate, stereoisomer, or prodrug thereof, wherein:
Y is —C(O)—, —CH₂, —CH₂C(O)—, —C(O)CH₂—, or SO₂;
Z is —H, —C(O)R³, —(C₀₋₁-alkyl)-SO₂—(C₁₋₄-alkyl), —C₁₋₈-alkyl, —CH₂OH, CH₂(O)(C₁₋₈-alkyl) or —CN;
R¹ and R² are each independently —CHF₂, —C₁₋₈-alkyl, —C₃₋₁₈-cycloalkyl, or —(C₁₋₁₀-alkyl)(C₃₋₁₈-cycloalkyl), and at least one of R₁ and R₂ is CHF₂;
R³ is —NR⁴R⁵, -alkyl, —OH, —O-alkyl, phenyl, benzyl, substituted phenyl, or substituted benzyl;
R⁴ and R⁵ are each independently —H, —C₁₋₈-alkyl, —OH, or —OC(O)R⁶;
R⁶ is —C₁₋₈-alkyl, -amino(C₁₋₈-alkyl), -phenyl, -benzyl, or -aryl;
X¹, X², X³, and X⁴ are each independently —H, -halogen, -nitro, —NH₂, —CF₃, —(C₀₋₄-alkyl)-(C₃₋₆-cycloalkyl), (C₀₋₄-alkyl)-NR⁷R⁸, (C₀₋₄-alkyl)-N(H)C(O)—(R⁸), (C₀₋₄-alkyl)-N(H)C(O)N(R⁷R⁸), (C₀₋₄-alkyl)-N(H)C(O)O(R⁷R⁸), (C₀₋₄-alkyl)-OR⁸, (C₀₋₄-alkyl)-pyrrolyl, (C₀₋₄-alkyl)-oxadiazolyl, or (C₀₋₄-alkyl)-triazolyl, or two of X¹, X², X³, and X⁴ may be joined together to form a cycloalkyl or heterocycloalkyl ring, (e.g., X¹ and X², X² and X³, X³ and X⁴, X¹ and X³, X² and X⁴, or X¹ and X⁴ may form a 3, 4, 5, 6, or 7 membered ring which may be aromatic, thereby forming a bicyclic system with the isoindolyl ring); and
R⁷ and R⁸ are each independently H, C₁₋₉-alkyl, C₃₋₆-cycloalkyl, (C₁₋₆-alkyl)-(C₃₋₆-cycloalkyl), (C₁₋₆-alkyl)-N(R⁷R⁸), (C₁₋₆-alkyl)-OR⁸, phenyl, benzyl, or aryl.

Other PDE4 modulators include the enantiomerically pure compounds disclosed in U.S. Pat. No. 6,962,940; international patent publication nos. WO 2003/080048 and WO 2003/080049; U.S. Pat. No. 7,312,241 to G. Muller et al.; and U.S. patent publication no. 2004/0167199A1, published Aug. 26, 2004, all of which are incorporated herein by reference. In certain embodiments, the compounds are an enantiomer of 2-[1-(3-ethoxy-4-methoxyphenyl)-2-methylsulfonylethyl]-4-acetylaminoisoindoline-1,3-dione and an enantiomer of 3-(3,4-dimethoxy-phenyl)-3-(1-oxo-1,3-dihydro-isoindol-2-yl)-propionamide.

In certain embodiments, the PDE4 modulators provided herein are 3-(3,4-dimethoxyphenyl)-3-(1-oxo-1,3-dihydro-isoindol-2-yl)-propionamide and cyclopropanecarboxylic acid {2-[1-(3-ethoxy-4-methoxy-phenyl)-2-methanesulfonyl-ethyl]-3-oxo-2,3-dihydro-1H-isoindol-4-yl}-amide, which are available from Celgene Corp., Warren, N.J. 3-(3,4-Dimethoxy-phenyl)-3-(1-oxo-1,3-dihydro-isoindol-2-yl)-propionamide has the following chemical structure:

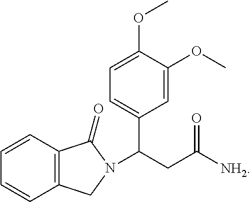 [SMILES: COc1ccc(C(CC(N)=O)N2Cc3ccccc3C2=O)cc1OC]

Other PDE4 modulators include, but are not limited to, the cycloalkyl amides and cycloalkyl nitriles of U.S. Pat. Nos. 5,728,844, 5,728,845, 5,968,945, 6,180,644 and 6,518,281, and WO 97/08143 and WO 97/23457, each of which is incorporated herein by reference. Representative compounds include those of formula:

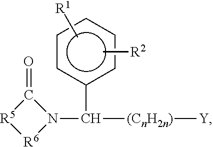

or a pharmaceutically acceptable salt, solvate, hydrate, clathrate, stereoisomer, or prodrug thereof, wherein:
one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, or R³—X—;
R³ is monocycloalkyl, bicycloalkyl, or benzocycloalkyl of up to 18 carbon atoms;
X is a carbon-carbon bond, —CH₂—, or —O—;
R⁵ is (i) o-phenylene, unsubstituted or substituted with 1 to 3 substituents each selected independently from nitro, cyano, halo, trifluoromethyl, carbo(lower)alkoxy, acetyl, or carbamoyl, unsubstituted or substituted with lower alkyl, acetoxy, carboxy, hydroxy, amino, lower alkylamino, lower acylamino, and lower alkoxy; (ii) a vicinally divalent residue of pyridine, pyrrolidine, imidazole, naphthalene, or thiophene, wherein the divalent bonds are on vicinal ring carbon atoms; (iii) a vicinally divalent cycloalkyl or cycloalkenyl of 4-10 carbon atoms, unsubstituted or substituted with 1 to 3 substituents each selected independently from the group consisting of nitro, cyano, halo, trifluoromethyl, carbo(lower)alkoxy, acetyl, carbamoyl, acetoxy, carboxy, hydroxy, amino, lower alkylamino, lower alkyl, lower alkoxy, or phenyl; (iv) vinylene di-substituted with lower alkyl; or (v) ethylene, unsubstituted or monosubstituted or disubstituted with lower alkyl;
R⁶ is —CO—, —CH₂—, or —CH₂CO—;
Y is —COZ, —C≡N, —OR⁸, lower alkyl, or aryl;
Z is —NH₂, —OH, —NHR, —R⁹, or —OR⁹
R⁸ is hydrogen or lower alkyl;
R⁹ is lower alkyl or benzyl; and
n has a value of 0, 1, 2, or 3.

In one embodiment, one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, or R³—X—;

R³ is monocycloalkyl of up to 10 carbon atoms, polycycloalkyl of up to 10 carbon atoms, or benzocyclic alkyl of up to 10 carbon atoms;

### X is —CH₂— or —O—;

R⁵ is (i) the vicinally divalent residue of pyridine, pyrrolidine, imidazole, naphthalene, or thiophene, wherein the two bonds of the divalent residue are on vicinal ring carbon atoms;
(ii) a vicinally divalent cycloalkyl of 4-10 carbon atoms, unsubstituted or substituted with 1 to 3 substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and phenyl;
(iii) di-substituted vinylene, substituted with nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;
(iv) ethylene, unsubstituted or substituted with 1 to 2 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, and halo;
R⁶ is —CO—, —CH₂—, or —CH₂CO—;
Y is —COX, —C≡N, —OR⁸, alkyl of 1 to 5 carbon atoms, or aryl;
X is —NH₂, —OH, —NHR, —R⁹, —OR⁹, or alkyl of 1 to 5 carbon atoms;
R⁸ is hydrogen or lower alkyl;
R⁹ is alkyl or benzyl; and,
n has a value of 0, 1, 2, or 3.

In another embodiment, one of R¹ and R² is R³—X— and the other is hydrogen, nitro, cyano, trifluoromethyl, carbo(lower)alkoxy, acetyl, carbamoyl, acetoxy, carboxy, hydroxy, amino, lower alkyl, lower alkoxy, halo, HF₂CO, F₃CO, or R³—X—;

R³ is monocycloalkyl, bicycloalkyl, benzocyclo alkyl of up to 18 carbon atoms, tetrahydropyran, or tetrahydrofuran;
X is a carbon-carbon bond, —CH₂—, —O—, or —N═;
R⁵ is (i) o-phenylene, unsubstituted or substituted with 1 to 3 substituents each selected independently from nitro, cyano, halo, trifluoromethyl, carbo(lower)alkoxy, acetyl, or carbamoyl, unsubstituted or substituted with lower alkyl, acetoxy, carboxy, hydroxy, amino, lower alkylamino, lower acylamino, and lower alkoxy; (ii) a vicinally divalent residue of pyridine, pyrrolidine, imidazole, naphthalene, or thiophene, wherein the divalent bonds are on vicinal ring carbon atoms; (iii) a vicinally divalent cycloalkyl or cycloalkenyl of 4-10 carbon atoms, unsubstituted or substituted with 1 or more substituents each selected independently from the group consisting of nitro, cyano, halo, trifluoromethyl, carbo(lower)alkoxy, acetyl, carbamoyl, acetoxy, carboxy, hydroxy, amino, lower alkylamino, lower alkyl, lower alkoxy, and phenyl; (iv) vinylene di-substituted with lower alkyl; or (v) ethylene, unsubstituted or monosubstituted or disubstituted with lower alkyl;
R⁶ is —CO—, —CH₂—, or —CH₂CO—;
Y is —COX, —C≡N, —OR⁸, alkyl of 1 to 5 carbon atoms, or aryl;
X is —NH₂, —OH, —NHR, —R⁹, —OR⁹, or alkyl of 1 to 5 carbon atoms;
R⁸ is hydrogen or lower alkyl;
R⁹ is alkyl or benzyl; and,
n has a value of 0, 1, 2, or 3.

Other representative compounds include those of formula:

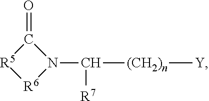

or a pharmaceutically acceptable salt, solvate, hydrate, clathrate, stereoisomer, or prodrug thereof, wherein:
Y is —C≡N or CO(CH₂)ₘCH₃;
m is 0, 1, 2, or 3;
R⁵ is (i) o-phenylene, unsubstituted or substituted with 1 to 3 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, and halo; (ii) the divalent residue of pyridine, pyrrolidine, imidizole, naphthalene, or thiophene, wherein the divalent bonds are on vicinal ring carbon atoms; (iii) a divalent cycloalkyl of 4-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl and halo; (iv) di-substituted vinylene, substituted with nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; or (v) ethylene, unsubstituted or substituted with 1 to 2 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, and halo;
R⁶ is —CO—, —CH₂—, —CH₂CO—, or —SO₂—;
R⁷ is (i) straight or branched alkyl of 1 to 12 carbon atoms; (ii) cyclic or bicyclic alkyl of 1 to 12 carbon atoms; (iii) pyridyl; (iv) phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, straight, branched, cyclic, or bicyclic alkyl of 1 to 10 carbon atoms, straight, branched, cyclic, or bicyclic alkoxy of 1 to 10 carbon atoms, CH₂R where R is a cyclic or bicyclic alkyl of 1 to 10 carbon atoms, and halo; (v) benzyl substituted with one to three substituents each selected independently from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 10 carbon atoms, and halo; (vi) naphthyl; or (vii) benzyloxy; and n has a value of 0, 1, 2, or 3.

In another embodiment, the PDE4 modulators include those of formula:

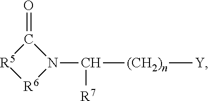

or a pharmaceutically acceptable salt, solvate, hydrate, clathrate, stereoisomer, or prodrug thereof, wherein:
R⁵ is (i) the divalent residue of pyridine, pyrrolidine, imidizole, naphthalene, or thiophene, wherein the divalent bonds are on vicinal ring carbon atoms; (ii) a divalent cycloalkyl of 4-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl and halo; (iii) di-substituted vinylene, substituted with nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo; or (iv) ethylene, unsubstituted or substituted with 1 to 2 substituents each selected independently from nitro, cyano, trifluoromethyl, carbethoxy, carbomethoxy, carbopropoxy, acetyl, carbamoyl, carbamoyl substituted with an alkyl of 1 to 3 carbon atoms, acetoxy, carboxy, hydroxy, amino, amino substituted with an alkyl of 1 to 3 carbon atoms, alkyl of 1 to 4 carbon atoms, alkoxy of 1 to 4 carbon atoms, or halo;
R⁶ is —CO—, —CH₂—, —CH₂CO—, or —SO₂—;
R⁷ is (i) cyclic or bicyclic alkyl of 4 to 12 carbon atoms; (ii) pyridyl; (iii) phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, straight, branched, cyclic, or bicyclic alkyl of 1 to 10 carbon atoms, straight, branched, cyclic, or bicyclic alkoxy of 1 to 10 carbon atoms, CH₂R where R is a cyclic or bicyclic alkyl of 1 to 10 carbon atoms, or halo; (iv) benzyl substituted with one to three substituents each selected independently from the group consisting of nitro, cyano, trifluoromethyl, carbethoxy, carbomethoxy, carbopropoxy, acetyl, carbamoyl, acetoxy, carboxy, hydroxy, amino, alkyl of 1 to 4 carbon atoms, alkoxy of 1 to 10 carbon atoms, and halo; (v) naphthyl; or (vi) benzyloxy; and
Y is COX, —C≡N, OR⁸, alkyl of 1 to 5 carbon atoms, or aryl;
X is —NH₂, —OH, —NHR, —R⁹, —OR⁹, or alkyl of 1 to 5 carbon atoms;
R⁸ is hydrogen or lower alkyl;
R⁹ is alkyl or benzyl; and
n has a value of 0, 1, 2, or 3.

Other PDE4 modulators include, but are not limited to, the aryl amides (for example, an embodiment being N-benzoyl-3-amino-3-(3′,4′-dimethoxyphenyl)-propanamide) of U.S. Pat. Nos. 5,801,195, 5,736,570, 6,046,221 and 6,284,780, each of which is incorporated herein by reference. Representative compounds include those of formula:

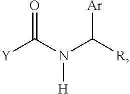

or a pharmaceutically acceptable salt, solvate, hydrate, clathrate, stereoisomer, or prodrug thereof, wherein:
Ar is (i) straight, branched, or cyclic, unsubstituted alkyl of 1 to 12 carbon atoms; (ii) straight, branched, or cyclic, substituted alkyl of 1 to 12 carbon atoms; (iii) phenyl; (iv) phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and halo; (v) heterocycle; or (vi) heterocycle substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;
R is —H, alkyl of 1 to 10 carbon atoms, CH₂OH, CH₂CH₂OH, or CH₂COZ where Z is alkoxy of 1 to 10 carbon atoms, benzyloxy, or NHR¹ where R¹ is H or alkyl of 1 to 10 carbon atoms; and
Y is i) a phenyl or heterocyclic ring, unsubstituted or substituted one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo; or ii) naphthyl.

Other examples of the compounds include those of formula:

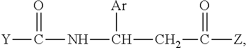

or a pharmaceutically acceptable salt, solvate, hydrate, clathrate, stereoisomer, or prodrug thereof, wherein:
Ar is 3,4-disubstituted phenyl where each substituent is selected independently of the other from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;
Z is alkoxy of 1 to 10 carbon atoms, benzyloxy, amino, or alkylamino of 1 to 10 carbon atoms; and
Y is (i) a phenyl, unsubstituted or substituted with one or more substituents each selected, independently one from the other, from the group consisting of nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, or (ii) naphthyl.

Other PDE4 modulators include, but are not limited to, the imide/amide ethers and alcohols (for example, 3-phthalimido-3-(34′-dimethoxyphenyl) propan-1-ol) disclosed in U.S. Pat. No. 5,703,098, which is incorporated herein by reference. Examples include compounds the formula:

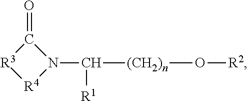

or a pharmaceutically acceptable salt, solvate, hydrate, clathrate, stereoisomer, or prodrug thereof, wherein:
R¹ is (i) straight, branched, or cyclic, unsubstituted alkyl of 1 to 12 carbon atoms; (ii) straight, branched, or cyclic, substituted alkyl of 1 to 12 carbon atoms; (iii) phenyl; or (iv) phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, acylamino, alkylamino, di(alkyl) amino, alkyl of 1 to 10 carbon atoms, cycloalkyl of 3 to 10 carbon atoms, bicycloalkyl of 5 to 12 carbon atoms, alkoxy of 1 to 10 carbon atoms, cycloalkoxy of 3 to 10 carbon atoms, bicycloalkoxy of 5 to 12 carbon atoms, and halo;
R² is hydrogen, alkyl of 1 to 8 carbon atoms, benzyl, pyridylmethyl, or alkoxymethyl;
R³ is (i) ethylene, (ii) vinylene, (iii) a branched alkylene of 3 to 10 carbon atoms, (iv) a branched alkenylene of 3 to 10 carbon atoms, (v) cycloalkylene of 4 to 9 carbon atoms unsubstituted or substituted with one or more substituents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (vi) cycloalkenylene of 4 to 9 carbon atoms unsubstituted or substituted with one or more substituents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (vii) o-phenylene unsubstituted or substituted with one or more substituents each selected independently from the group consisting of nitro, cyano, trifluoromethyl, carbethoxy, carbomethoxy, carbopropoxy, acetyl, carbamoyl, acetoxy, carboxy, hydroxy, amino, amino substituted with alkyl of 1 to 6 carbon atoms, amino substituted with acyl of 1 to 6 carbon atoms, alkyl of 1 to 10 carbon atoms, alkoxy of 1 to 12 carbon atoms, and halo, (viii) naphthyl, or (ix) pyridyl;
R⁴ is —CX—, —CH₂— or —CH₂CX—;

### X is O or S; and

n is 0, 1, 2, or 3.

Other PDE4 modulators include, but are not limited to, the succinimides and maleimides (for example methyl 3-(3′,4′,5′6′-petrahydrophthalimdo)-3-(3″,4″-dimethoxyphenyl)propionate) disclosed in U.S. Pat. No. 5,658,940, which is incorporated herein by reference. Examples include compounds of formula:

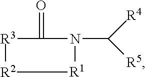

or a pharmaceutically acceptable salt, solvate, hydrate, clathrate, stereoisomer, or prodrug thereof, wherein:
R¹ is —CH₂—, —CH₂CO—, or —CO—;
R² and R³ taken together are (i) ethylene unsubstituted or substituted with alkyl of 1-10 carbon atoms or phenyl, (ii) vinylene substituted with two substituents each selected, independently of the other, from the group consisting of alkyl of 1-10 carbon atoms and phenyl, or (iii) a divalent cycloalkyl of 5-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl unsubstituted or substituted with alkyl of 1-3 carbon atoms, acetoxy, carboxy, hydroxy, amino, substituted amino, alkyl of 1 to 10 carbon atoms, alkoxy of 1 to 10 carbon atoms, norbornyl, phenyl and halo;
R⁴ is (i) straight or branched unsubstituted alkyl of 4 to 8 carbon atoms; (ii) cycloalkyl or bicycloalkyl of 5-10 carbon atoms, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, branched, straight or cyclic alkyl of 1 to 10 carbon atoms, alkoxy of 1 to 10 carbon atoms, phenyl and halo; (iii) phenyl substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, cycloalkyl or bicyctoalkyl of 3 to 10 carbon atoms, cycloalkoxy or bicycloalkoxy of 3 to 10 carbon atoms, phenyl and halo; (iv) pyridine or pyrrolidine, unsubstituted or substituted with one or more substituents each selected independently of the other from the group consisting of nitro, cyano, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, phenyl and halo;
R⁵ is —COX, —CN, —CH₂COX, alkyl of 1 to 5 carbon atoms, aryl, —CH₂OR, —CH₂ aryl, or —CH₂OH,
X is NH₂, OH, NHR, or OR⁶,
R is lower alkyl; and
R⁶ is alkyl or benzyl.

Other PDE4 modulators include, but are not limited to, substituted imides (for example, 2-phthalimido-3-(3′,4′-dimethoxyphenyl) propane) disclosed in U.S. Pat. No. 6,429,221, which is incorporated herein by reference. Examples include compounds of the formula:

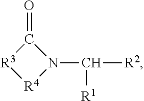

or a pharmaceutically acceptable salt, solvate, hydrate, clathrate, stereoisomer, or prodrug thereof, wherein:
R¹ is (i) straight, branched, or cyclic alkyl of 1 to 12 carbon atoms; (ii) phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, straight or branched alkyl of 1 to 10 carbon atoms, alkoxy of 1 to 10 carbon atoms, and halo; (iii) benzyl or benzyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo, or (iv) —Y-Ph where Y is a straight, branched, or cyclic alkyl of 1 to 12 carbon atoms and Ph is phenyl or phenyl substituted with one or more substituents each selected independently of the other from nitro, cyano, trifluoromethyl, carbethoxy, carbomethoxy, carbopropoxy, acetyl, carbamoyl, acetoxy, carboxy, hydroxy, amino, alkyl of 1 to 10 carbon atoms, alkoxy of 1 to 10 carbon atoms, and halo;
R² is —H, a branched or unbranched alkyl of 1 to 10 carbon atoms, phenyl, pyridyl, heterocycle, —CH₂-aryl, or —CH₂-heterocycle;
R³ is i) ethylene; ii) vinylene; iii) a branched alkylene of 3 to 10 carbon atoms; iv) a branched alkenylene of 3 to 10 carbon atoms; v) cycloalkylene of 4 to 9 carbon atoms unsubstituted or substituted with 1 to 2 substituents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy of 1 to 4 carbon atoms, and halo; vi) cycloalkenylene of 4 to 9 carbon atoms unsubstituted or substituted with 1 to 2 substituents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy of 1 to 4 carbon atoms, or halo; or vii) o-phenylene unsubstituted or substituted with 1 to 2 substituents each selected independently from nitro, cyano, trifluoromethyl, carbethoxy, carbomethoxy, carbopropoxy, acetyl, carbamoyl, acetoxy, carboxy, hydroxy, amino, substituted amino, alkyl of 1 to 4 carbon atoms, alkoxy 1 to 4 carbon atoms, and halo;
R⁴ is —CX, or —CH₂—; and

### X is O or S.

Other PDE4 modulators include, but are not limited to, substituted 1,3,4-oxadiazoles (for example, 2-[1-(3-cyclopentyloxy-4-methoxyphenyl)-2-(1,3,4-oxadiazole-2-yl)ethyl]-5-methylisoindoline-1,3-dione) disclosed in U.S. Pat. No. 6,326,388, which is incorporated herein by reference. Examples include compounds of formula:

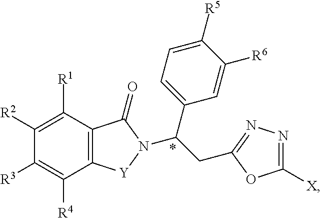

or a pharmaceutically acceptable salt, solvate, hydrate, clathrate, stereoisomer, or prodrug thereof, wherein:
the carbon atom designated* constitutes a center of chirality;
Y is C═O, CH₂, SO₂ or CH₂C═O;
X is hydrogen, or alkyl of 1 to 4 carbon atoms;
each of R¹, R², R³, and R⁴, independently of the others, is hydrogen, halo, trifluoromethyl, acetyl, alkyl of 1 to 8 carbon atoms, alkoxy of 1 to 4 carbon atoms, nitro, cyano, hydroxy, —CH₂NR⁸R⁹, —(CH₂)₂NR⁸R⁹, or —NR⁸R⁹ or
any two of R¹, R², R³, and R⁴ on adjacent carbon atoms, together with the depicted benzene ring are naphthylidene, quinoline, quinoxaline, benzimidazole, benzodioxole or 2-hydroxybenzimidazole;
each of R⁵ and R⁶, independently of the other, is hydrogen, alkyl of 1 to 4 carbon atoms, alkoxy of 1 to 6 carbon atoms, cyano, benzocycloalkoxy, cycloalkoxy of up to 18 carbon atoms, bicyloalkoxy of up to 18 carbon atoms, tricylcoalkoxy of up to 18 carbon atoms, or cycloalkylalkoxy of up to 18 carbon atoms;
each of R⁸ and R⁹, taken independently of the other is hydrogen, straight or branched alkyl of 1 to 8 carbon atoms, phenyl, benzyl, pyridyl, or pyridylmethyl; or one of R⁸ and R⁹ is hydrogen and the other is —COR¹⁰, or —SO₂R¹⁰; or R⁸ and R⁹ taken together are tetramethylene, pentamethylene, hexamethylene, —CH═NCH═CH—, or —CH₂CH₂X¹CH₂CH₂— in which X¹ is —O—, —S—, or —NH—;
R¹⁰ is hydrogen, alkyl of 1 to 8 carbon atoms, cycloalkyl, cycloalkylmethyl of up to 6 carbon atoms, phenyl, pyridyl, benzyl, imidazolylmethyl, pyridylmethyl, NR¹¹R¹², CH₂R¹⁴R¹⁵, or NR¹¹R¹²;
R¹⁴ and R¹⁵, independently of each other, are hydrogen, methyl, ethyl, or propyl; and
R¹¹ and R¹², independently of each other, are hydrogen, alkyl of 1 to 8 carbon atoms, phenyl, or benzyl.

In certain embodiments, the compounds include those of formula:

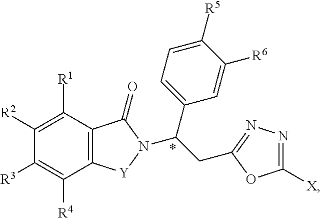

or a pharmaceutically acceptable salt, solvate, hydrate, clathrate, stereoisomer, or prodrug thereof, wherein:
the carbon atom designated* constitutes a center of chirality;
Y is C═O, CH₂, SO₂ or CH₂C═O;
X is hydrogen, or alkyl of 1 to 4 carbon atoms;
(i) each of R¹, R², R³, and R⁴, independently of the others, is hydrogen, halo, trifluoromethyl, acetyl, alkyl of 1 to 8 carbon atoms, alkoxy of 1 to 4 carbon atoms, nitro, cyano, hydroxy, —CH₂NR⁸R⁹, —(CH₂)₂NR⁸R⁹, or —NR⁸R⁹; or
(ii) any two of R¹, R², R³, and R⁴ on adjacent carbon atoms, together with the depicted benzene ring to which they are bound are naphthylidene, quinoline, quinoxaline, benzimidazole, benzodioxole or 2-hydroxybenzimidazole;
each of R⁵ and R⁶, independently of the other, is hydrogen, alkyl of 1 to 4 carbon atoms, alkoxy of 1 to 6 carbon atoms, cyano, benzocycloalkoxy, cycloalkoxy of up to 18 carbon atoms, bicyloalkoxy of up to 18 carbon atoms, tricylcoalkoxy of up to 18 carbon atoms, or cycloalkylalkoxy of up to 18 carbon atoms;
(i) each of R⁸ and R⁹, independently of the other, is hydrogen, alkyl of 1 to 8 carbon atoms, phenyl, benzyl, pyridyl, or pyridylmethyl; or
(ii) one of R⁸ and R⁹ is hydrogen and the other is —COR¹⁰ or —SO₂R¹⁰, in which R¹⁰ is hydrogen, alkyl of 1 to 8 carbon atoms, cycloalkyl, cycloalkylmethyl of up to 6 carbon atoms, phenyl, pyridyl, benzyl, imidazolylmethyl, pyridylmethyl, or NR¹¹R¹², or CH₂NR¹⁴R¹⁵, wherein R¹¹ and R¹², independently of each other, are hydrogen, alkyl of 1 to 8 carbon atoms, phenyl, or benzyl and R¹⁴ and R¹⁵, independently of each other, are hydrogen, methyl, ethyl, or propyl; or
(iii) R⁸ and R⁹ taken together are tetramethylene, pentamethylene, hexamethylene, —CH═NCH═CH—, or —CH₂CH₂X¹CH₂CH₂,— in which X¹ is —O—, —S—, or —NH—.

Other PDE4 modulators include, but are not limited to, cyano and carboxy derivatives of substituted styrenes (for example, 3,3-bis-(3,4-dimethoxyphenyl) acrylonitrile) disclosed in U.S. Pat. Nos. 5,929,117, 6,130,226, 6,262,101 and 6,479,554, each of which is incorporated herein by reference. Examples include compounds of formula:

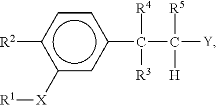

or a pharmaceutically acceptable salt, solvate, hydrate, clathrate, stereoisomer, or prodrug thereof, wherein:
(a) X is —O— or —(CₙH₂ₙ)— in which n has a value of 0, 1, 2, or 3, and R¹ is alkyl of one to 10 carbon atoms, monocycloalkyl of up to 10 carbon atoms, polycycloalkyl of up to 10 carbon atoms, or benzocyclic alkyl of up to 10 carbon atoms; or
(b) X is —CH═ and R¹ is alkylidene of up to 10 carbon atoms, monocycloalkylidene of up to 10 carbon atoms, or bicycloalkylidene of up to 10 carbon atoms;
R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkylidenemethyl, lower alkoxy, or halo;
R³ is (i) phenyl, unsubstituted or substituted with 1 or more substituents each selected independently from nitro, cyano, halo, trifluoromethyl, carbethoxy, carbomethoxy, carbopropoxy, acetyl, carbamoyl, carbamoyl substituted with alkyl of 1 to 3 carbon atoms, acetoxy, carboxy, hydroxy, amino, amino substituted with an alkyl of 1 to 5 carbon atoms, alkyl of up to 10 carbon atoms, cycloalkyl of up to 10 carbon atoms, alkoxy of up to 10 carbon atoms, cycloalkoxy of up to 10 carbon atoms, alkylidenemethyl of up to 10 carbon atoms, cycloalkylidenemethyl of up to 10 carbon atoms, phenyl, and methylenedioxy; (ii) pyridine, substituted pyridine, pyrrolidine, imidizole, naphthalene, or thiophene; (iii) cycloalkyl of 4-10 carbon atoms, unsubstituted or substituted with 1 or more substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, and alkoxy of 1 to 10 carbon atoms, phenyl; each of R⁴ and R⁵ taken individually is hydrogen; or R⁴ and R⁵ taken together are a carbon-carbon bond;
Y is —COZ, —C≡N, or lower alkyl of 1 to 5 carbon atoms;
Z is —OH, —NR⁶R⁶, —R⁷, or —OR⁷; R⁶ is hydrogen or lower alkyl; and R⁷ is alkyl or benzyl.

In some embodiments, PDE4 modulators include compounds of formula:

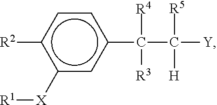

or a pharmaceutically acceptable salt, solvate, hydrate, clathrate, stereoisomer, or prodrug thereof, wherein:
(a) X is —O— or —(CₙH₂ₙ)— in which n has a value of 0, 1, 2, or 3, and R¹ is alkyl of one to 10 carbon atoms, monocycloalkyl of up to 10 carbon atoms, polycycloalkyl of up to 10 carbon atoms, or benzocyclic alkyl of up to 10 carbon atoms; or
(b) X is —CH═ and R¹ is alkylidene of up to 10 carbon atoms, monocycloalkylidene of up to 10 carbon atoms, or bicycloalkylidene of up to 10 carbon atoms;
R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkylidenemethyl, lower alkoxy, or halo;
R³ is pyrrolidine, imidazole or thiophene unsubstituted or substituted with 1 or more substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and phenyl; each of R⁴ and R⁵ taken individually is hydrogen; or R⁴ and R⁵ taken together are a carbon-carbon bond;
Y is —COZ, —C≡N, or lower alkyl of 1 to 5 carbon atoms;
Z is —OH, —NR⁶R⁶, —R⁷, or —OR⁷; R⁶ is hydrogen or lower alkyl; and R⁷ is alkyl or benzyl.

In some embodiments, provided herein are compounds of the formula:

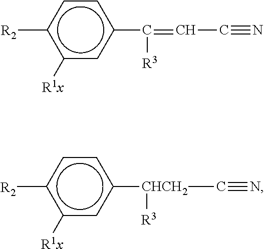

or a pharmaceutically acceptable salt, solvate, hydrate, clathrate, stereoisomer, or prodrug thereof, wherein:
(a) X is —O— or —(CₙH₂ₙ)— in which n has a value of 0, 1, 2, or 3, and R¹ is alkyl of up to 10 carbon atoms, monocycloalkyl of up to 10 carbon atoms, polycycloalkyl of up to 10 carbon atoms, or benzocyclic alkyl of up to 10 carbon atoms, or
(b) X is —CH═, and R¹ is alkylidene of up to 10 carbon atoms or monocycloalkylidene of up to 10 carbon atoms;
R² is hydrogen, nitro, cyano, trifluoromethyl, carbethoxy, carbomethoxy, carbopropoxy, acetyl, carbamoyl, acetoxy, carboxy, hydroxy, amino, lower alkyl, lower alkoxy, or halo; and
R³ is (i) phenyl or naphthyl, unsubstituted or substituted with 1 or more substituents each selected independently from nitro, cyano, halo, trifluoromethyl, carbethoxy, carbomethoxy, carbopropoxy, acetyl, carbamoyl, or carbamoyl substituted with alkyl of 1 to 3 carbon atoms, acetoxy, carboxy, hydroxy, amino, amino substituted with an alkyl of 1 to 5 carbon atoms, alkoxy and cycloalkoxy of 1 to 10 carbon atoms; or (ii) cycloalkyl of 4 to 10 carbon atoms, unsubstituted or substituted with one or more substituents each selected independently from the group consisting of nitro, cyano, halo, trifluoromethyl, carbethoxy, carbomethoxy, carbopropoxy, acetyl, carbamoyl, acetoxy, carboxy, hydroxy, amino, substituted amino, alkyl of 1 to 10 carbon atoms, alkoxy of 1 to 10 carbon atoms, and phenyl.

In one embodiment, the compound is of formula:

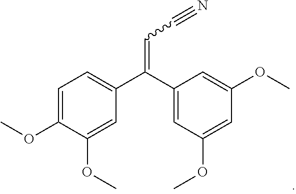

Other PDE4 modulators include, but are not limited to, isoindoline-1-one and isoindoline-1,3-dione substituted in the 2-position with an α-(3,4-disubstituted phenyl)alkyl group and in the 4- and/or 5-position with a nitrogen-containing group disclosed in WO 01/34606 and U.S. Pat. No. 6,667,316, which are incorporated herein by reference. Examples include compounds of formula:

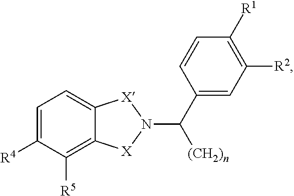

or a pharmaceutically acceptable salt, solvate, hydrate, clathrate, stereoisomer, or prodrug thereof, wherein:
one of X and X′ is ═C═O or ═SO₂, and the other of X and X′ is ═C═O, ═CH₂, ═SO₂ or ═CH₂C═O;
n is 1, 2 or 3;
R¹ and R² are each independently (C₁-C₄)alkyl, (C₁-C₄)alkoxy, cyano, (C₃-C₁₈)cycloalkyl, (C₃-C₁₈)cycloalkoxy, or (C₃-C₁₈)cycloalkyl-methoxy;
R³ is SO₂—Y, COZ, CN or (C₁-C₆)hydroxyalkyl, wherein:
Y is (C₁-C₆)alkyl, benzyl or phenyl;
Z is —NR⁶R⁷, (C₁-C₆)alkyl, benzyl or phenyl;
R⁶ is H, (C₁-C₄)alkyl, (C₃-C₁₈)cycloalkyl, (C₂-C₅)alkanoyl, benzyl or phenyl, each of which may be optionally substituted with halo, amino or (C₁-C₄)alkyl-amino;
R⁷ is H or (C₁-C₄)alkyl;
R⁴ and R⁵ are taken together to provide —NH—CH₂—R⁸—, NH—CO—R⁸—, or —N═CH—R⁸—, wherein:
R⁸ is CH₂, O, NH, CH═CH, CH═N, or N═CH; or
one of R⁴ and R⁵ is H, and the other of R₄ and R₅ is imidazoyl, pyrrolyl, oxadiazolyl, triazolyl, or a structure of formula (A),

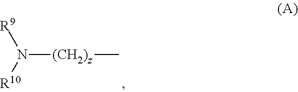

wherein:
z is 0 or 1;
R⁹ is: H; (C₁-C₄)alkyl, (C₃-C₁₈)cycloalkyl, (C₂-C₅)alkanoyl, or (C₄-C₆)cycloalkanoyl, optionally substituted with halo, amino, (C₁-C₄)alkyl-amino, or (C₁-C₄)dialkyl-amino; phenyl; benzyl; benzoyl; (C₂-C₅)alkoxycarbonyl; (C₃-C₅)alkoxyalkylcarbonyl; N-morpholinocarbonyl; carbamoyl; N-substituted carbamoyl substituted with (C₁-C₄)alkyl; or methylsulfonyl; and
R¹⁰ is H, (C₁-C₄)alkyl, methylsulfonyl, or (C₃-C₅)alkoxyalkylcarbonyl; or
R⁹ and R¹⁰ are taken together to provide —CH═CH—CH═CH—, —CH═CH—N═CH—, or (C₁-C₂)alkylidene, optionally substituted with amino, (C₁-C₄)alkyl-amino, or (C₁-C₄)dialkyl-amino; or;
R⁴ and R⁵ are both structures of formula (A).

In one embodiment, z is not 0 when (i) R³ is —SO₂—Y, —COZ, or —CN and (ii) one of R⁴ or R⁵ is hydrogen. In another embodiment, R⁹ and R¹⁰, taken together, are —CH═CH—CH═CH—, —CH═CH—N═CH—, or (C₁-C₂)alkylidene substituted by amino, (C₁-C₄)alkyl-amino, or (C₁-C₄)dialkyl-amino. In another embodiment, R⁴ and R⁵ are both structures of formula (A).

In some embodiments, compounds include those of formula:

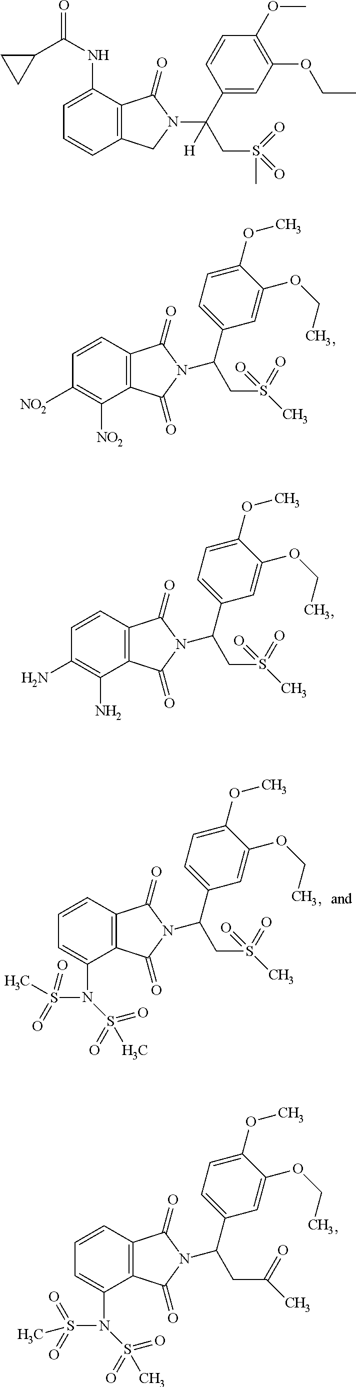

or a pharmaceutically acceptable salt, solvate, hydrate, clathrate, stereoisomer, or prodrug thereof.

Further examples include, but are not limited to: 2-[1-(3-Ethoxy-4-methoxyphenyl)-2-methylsulfonylethyl]-4,5-dinitroisoindoline-1,3-dione; 2-[1-(3-Ethoxy-4-methoxyphenyl)-2-methylsulfonylethyl]-4,5-diaminoisoindoline-1,3-dione; 7-[1-(3-Ethoxy-4-methoxyphenyl)-2-methylsulfonylethyl]-3-pyrrolino[3,4-e]benzimidazole-6,8-dione; 7-[1-(3-Ethoxy-4-methoxyphenyl)-2-methylsulfonylethyl]hydro-3-pyrrolino[3,4-e]benzimidazole-2,6,8-trione; 2-[1-(3-Ethoxy-4-methoxyphenyl)-2-methylsulfonylethyl]-3-pyrrolino[3,4-f]quinoxaline-1,3-dione; Cyclopropyl-N-{2-[1-(3-ethoxy-4-methoxyphenyl)-2-methylsulfonylethyl]-1,3-dioxoisoindolin-4-yl}carboxamide; 2-Chloro-N-{2-[1-(3-ethoxy-4-methoxyphenyl)-2-methylsulfonylethyl]-1,3-dioxoisoindolin-4-yl}acetamide; 2-Amino-N-{2-[1-(3-ethoxy-4-methoxyphenyl)-2-methylsulfonylethyl]-1,3-dioxoisoindolin-4-yl}acetamide; 2-N,N-Dimethylamino-N-{2-[-(3-ethoxy-4-methoxyphenyl)-2-methylsulfonylethyl]-1,3-dioxoisoindolin-4-yl}acetamide; N-{2-[1-(3-ethoxy-4-methoxyphenyl)-2-methylsulfonylethyl]-1,3-dioxoisoindolin-4-yl}-2,2,2-trifluoroacetamide; N-{2-[1-(3-Ethoxy-4-methoxyphenyl)-2-methylsulfonylethyl]-1,3-dioxoisoindolin-4-yl}methoxycarboxamide; 4-[1-Aza-2-(dimethylamino)vinyl]-2-[1-(3-ethoxy-4-methoxyphenyl)-2-methylsulfonylethyl]isoindoline-1,3-dione; 4-[1-Aza-2-(dimethylamino)prop-1-enyl]-2-[1-(3-ethoxy-4-methoxyphenyl)-2-methylsulfonylethyl]isoindoline-1,3-dione; 2-[1-(3-Ethoxy-4-methoxyphenyl)-2-methylsulfonylethyl]-4-(5-methyl-1,3,4-oxadiazol-2-yl)isoindoline-1,3-dione; 2-[1-(3-Ethoxy-4-methoxyphenyl)-2-methylsulfonylethyl]-4-pyrrolylisoindoline-1,3-dione; 4-(Aminomethyl)-2-[1-(3-ethoxy-4-methoxyphenyl)-2-methylsulfonylethyl]-isoindoline-1,3-dione; 2-[1-(3-Ethoxy-4-methoxyphenyl)-2-methylsulfonylethyl]-4-(pyrrolylmethyl)isoindoline-1,3-dione; N-{2-[1-(3-ethoxy-4-methoxyphenyl)-3-hydroxybutyl]-1,3-dioxoisoindolin-4-yl}acetamide; N-{2-[1-(3-Ethoxy-4-methoxyphenyl)-3-oxobutyl]-1,3-dioxoisoindolin-4-yl}acetamide; N-{2-[1R-(3-ethoxy-4-methoxyphenyl)-3-hydroxybutyl]-1,3-dioxoisoindolin-4-yl}acetamide; N-{2-[1R-(3-ethoxy-4-methoxyphenyl)-3-oxobutyl]-1,3-dioxoisoindolin-4-yl}acetamide; N-{2-[1 S-(3-Ethoxy-4-methoxyphenyl)-3-hydroxybutyl]-1,3-dioxoisoindolin-4-yl}acetamide; N-{2-[1S-(3-ethoxy-4-methoxyphenyl)-3-oxobutyl]-1,3-dioxoisoindolin-4-yl}acetamide; 4-Amino-2-[1-(3-ethoxy-4-methoxyphenyl)-3-hydroxybutylisoindoline-1,3-dione; 4-Amino-2-[1-(3-ethoxy-4-methoxyphenyl)-3-oxobutyl]isoindoline-1,3-dione; 2-[1-(3-Ethoxy-4-methoxyphenyl)-3-oxobutyl]-4-pyrrolylisoindoline-1,3-dione; 2-Chloro-N-{2-[1-(3-ethoxy-4-methoxyphenyl)-3-oxobutyl]-1,3-dioxoisoindol-4-yl}acetamide; 2-(Dimethylamino)-N-{2-[1-(3-ethoxy-4-methoxyphenyl)-3-oxobutyl]-1,3-dioxoisoindolin-4-yl}acetamide; 4-Amino-2-[1R-(3-ethoxy-4-methoxyphenyl)-3-hydroxybutyl]isoindoline-1,3-dione; 4-Amino-2-[1R-(3-ethoxy-4-methoxyphenyl)-3-oxobutyl]isoindoline-1,3-dione; 2-[1R-(3-ethoxy-4-methoxyphenyl)-3-oxobutyl]-4-pyrrolylisoindoline-1,3-dione; 2-(Dimethylamino)-N-{2-[1R-(3-ethoxy-4-methoxyphenyl)-3-oxobutyl]-1,3-dioxoisoindolin-4-yl}acetamide; Cyclopentyl-N-{2-[1-(3-ethoxy-4-methoxyphenyl)-2-(methylsulfonyl)ethyl]-1,3-dioxoisoindolin-4-yl}carboxamide; 3-(Dimethylamino)-N-{2-[1-(3-ethoxy-4-methoxyphenyl)-2-(methylsulfonyl)ethyl]-1,3-dioxoisoindolin-4-yl}propanamide; 2-(Dimethylamino)-N-{2-[1-(3-ethoxy-4-methoxyphenyl)-2-(methylsulfonyl)ethyl]-1,3-dioxoisoindolin-4-yl}propanamide; N-{2-[(1R)-1-(3-ethoxy-4-methoxyphenyl)-2-(methylsulfonyl)ethyl]-1,3-dioxoisoindolin-4-yl}-2-(dimethylamino)acetamide; N-{2-[(1S)-1-(3-ethoxy-4-methoxyphenyl)-2-(methylsulfonyl)ethyl]-1,3-dioxoisoindolin-4-yl}-2-(dimethylamino)acetamide; 4-{3-[(Dimethylamino)methyl]pyrrolyl}-2-[1-(3-ethoxy-4-methoxyphenyl)-2-(methylsulfonyl)ethyl]isoindoline-1,3-dione; Cyclopropyl-N-{2-[(1S)-1-(3-ethoxy-4-methoxyphenyl)-2-(methylsulfonyl)ethyl]-1,3-dioxoisoindolin-4-yl}carboxamide; 2-[1-(3,4-dimethoxyphenyl)-2-(methylsulfonyl)ethyl]-4-pyrrolylisoindoline-1,3-dione; N-{2-[1-(3,4-dimethoxyphenyl)-2-(methylsulfonyl)ethyl]-1,3-dioxoisoindolin-4-yl}-2-(dimethylamino)acetamide; Cyclopropyl-N-{2-[1-(3,4-dimethoxyphenyl)-2-(methylsulfonyl)ethyl]-1,3-dioxoisoindolin-4-yl}carboxamide; Cyclopropyl-N-{2-[1-(3-ethoxy-4-methoxyphenyl)-2-(methylsulfonyl)ethyl]-3-oxoisoindolin-4-yl}carboxamide; 2-(Dimethylamino)-N-{2-[1-(3-ethoxy-4-methoxyphenyl)-2-(methylsulfonyl)ethyl]-3-oxoisoindolin-4-yl}acetamide; Cyclopropyl-N-{2-[(1S)-1-(3-ethoxy-4-methoxyphenyl)-2-(methylsulfonyl)ethyl]-3-oxoisoindolin-4-yl}carboxamide; Cyclopropyl-N-{2-[(1R)-1-(3-ethoxy-4-methoxyphenyl)-2-(methylsulfonyl)ethyl]-3-oxoisoindolin-4-yl}carboxamide; (3R)-3-[7-(Acetylamino)-1-oxoisoindolin-2-yl]-3-(3-ethoxy-4-methoxyphenyl)-N,N-dimethylpropanamide; (3R)-3-[7-(Cyclopropylcarbonylamino)-1-oxoisoindolin-2-yl]-3-(3-ethoxy-4-methoxyphenyl)-N,N-dimethylpropanamide; 3-{4-[2-(Dimethylamino)acetylamino]-1,3-dioxoisoindolin-2-yl}-3-(3-ethoxy-4-methoxyphenyl)-N,N-dimethylpropanamide; (3R)-3-[7-(2-Chloroacetylamino)-1-oxoisoindolin-2-yl]-3-(3-ethoxy-4-methoxy-phenyl)-N,N-dimethylpropanamide; (3R)-3-{4-[2-(dimethylamino)acetylamino]-1,3-dioxoisoindolin-2-yl}-3-(3-ethoxy-4-methoxyphenyl)-N,N-dimethylpropanamide; 3-(1,3-Dioxo-4-pyrrolylisoindolin-2-yl)-3-(3-ethoxy-4-methoxyphenyl)-N,N-dimethylpropanamide; 2-[1-(3-Ethoxy-4-methoxyphenyl)-2-(methylsulfonyl)ethyl]-4-(imidazolyl-methyl)isoindoline-1,3-dione; N-({2-[1-(3-Ethoxy-4-methoxyphenyl)-2-(methylsulfonyl)ethyl]-1,3-dioxoisoindolin-4-yl}methyl)acetamide; 2-Chloro-N-({2-[1-(3-ethoxy-4-methoxyphenyl)-2-(methylsulfonyl)ethyl]-1,3-dioxoisoindolin-4-yl}methyl)acetamide; 2-(Dimethylamino)-N-({2-[1-(3-ethoxy-4-methoxyphenyl)-2-(methylsulfonyl)ethyl]-1,3-dioxoisoindolin-4-yl}methyl)acetamide; 4-[Bis(methylsulfonyl)amino]-2-[1-(3-ethoxy-4-methoxyphenyl)-2-(methylsulfonyl)ethyl]isoindoline-1,3-dione; 2-[1-(3-Ethoxy-4-methoxyphenyl)-2-(methylsulfonyl)ethyl]-4-[(methylsulfonyl)amino]isoindoline-1,3-dione; N-{2-[1-(3-Ethoxy-4-methoxyphenyl)-3-hydroxypentyl]-1,3-dioxoisoindolin-4-yl}acetamide; N-{2-[1-(3-Ethoxy-4-methoxyphenyl)-3-oxopentyl]1,3-dioxoisoindolin-4-yl}acetamide; 2-[(1R)-1-(3-Ethoxy-4-methoxyphenyl)-3-hydroxybutyl]-4-(pyrrolylmethyl)isoindoline-1,3-dione; 2-[(1R)-1-(3-Ethoxy-4-methoxyphenyl)-3-oxobutyl]-4-(pyrrolylmethyl)isoindoline-1,3-dione; N-{2-[1-(3-Cyclopentyloxy-4-methoxyphenyl)-3-hydroxybutyl]-1,3-dioxoisoindolin-4-yl}acetamide; N-{2-[1-(3-Cyclopentyloxy-4-methoxyphenyl)-3-oxobutyl]-1,3-dioxoisoindolin-4-yl}acetamide; 2-[1-(3-Cyclopentyloxy-4-methoxyphenyl)-3-oxobutyl]-4-pyrrolylisoindoline-1,3-dione; 2-[1-(3,4-Dimethoxyphenyl)-3-oxobutyl]-4-[bis(methylsulfonyl)amino]isoindoline-1,3-dione; or a pharmaceutically acceptable salt, solvate, hydrate, clathrate, stereoisomer, or prodrug thereof

Still other PDE4 modulators include, but are not limited to, imido and amido substituted acylhydroxamic acids (for example, (3-(1,3-dioxoisoindoline-2-yl)-3-(3-ethoxy-4-methoxyphenyl) propanoylamino) propanoate disclosed in WO 01/45702 and U.S. Pat. No. 6,699,899, which are incorporated herein by reference. Examples include compounds of formula:

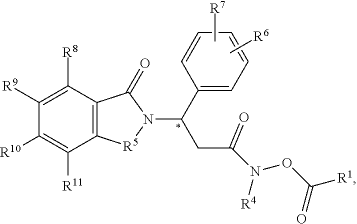

or a pharmaceutically acceptable salt, solvate, hydrate, clathrate, stereoisomer, or prodrug thereof, wherein:
the carbon atom designated * constitutes a center of chirality,
R⁴ is hydrogen or —(C═O)—R¹²,
each of R¹ and R¹², independently of each other, is alkyl of 1 to 6 carbon atoms, phenyl, benzyl, pyridyl methyl, pyridyl, imidazoyl, imidazolyl methyl, or CHR*(CH₂)ₙNR*R⁰,
wherein R*and R⁰, independently of the other, are hydrogen, alkyl of 1 to 6 carbon atoms, phenyl, benzyl, pyridyl methyl, pyridyl, imidazoyl or imidazolylmethyl, and n=0, 1, or 2;
R⁵ is C═O, CH₂, CH₂—CO—, or SO₂;
each of R⁶ and R⁷, independently of the other, is nitro, cyano, trifluoromethyl, carbethoxy, carbomethoxy, carbopropoxy, acetyl, carbamoyl, acetoxy, carboxy, hydroxy, amino, alkyl of 1 to 6 carbon atoms, alkoxy of 1 to 6 carbon atoms, cycloalkoxy of 3 to 8 carbon atoms, halo, bicycloalkyl of up to 18 carbon atoms, tricycloalkoxy of up to 18 carbon atoms, 1-indanyloxy, 2-indanyloxy, C₄-C₈-cycloalkylidenemethyl, or C₃-C₁₀-alkylidenemethyl;
each of R⁸, R⁹, R¹⁰, and R¹¹, independently of the others, is
(i) hydrogen, nitro, cyano, trifluoromethyl, carbethoxy, carbomethoxy, carbopropoxy, acetyl, carbamoyl, acetoxy, carboxy, hydroxy, amino, alkylamino, dialkylamino, acylamino, alkyl of 1 to 10 carbon atoms, or alkoxy of 1 to 10 carbon atoms, halo; or
(ii) one of R⁸, R⁹, R¹⁰, and R¹¹ is acylamino comprising a lower alkyl, and the remaining of R⁸, R⁹, R¹⁰, and R¹¹ are hydrogen; or
(iii) hydrogen if R⁸ and R⁹ taken together are benzo, quinoline, quinoxaline, benzimidazole, benzodioxole, 2-hydroxybenzimidazole, methylenedioxy, dialkoxy, or dialkyl; or
(iv) hydrogen if R¹⁰ and R¹¹, taken together are benzo, quinoline, quinoxaline, benzimidazole, benzodioxole, 2-hydroxybenzimidazole, methylenedioxy, dialkoxy, or dialkyl; or
(v) hydrogen if R⁹ and R¹⁰ taken together are benzo.

Still other PDE4 modulators include, but are not limited to, 7-amido-isoindolyl compounds disclosed in U.S. Pat. No. 7,034,052, which is incorporated herein by reference. Examples include compounds of formula:

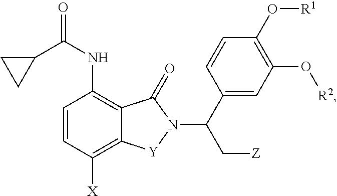

or a pharmaceutically acceptable salt, solvate, hydrate, clathrate, stereoisomer, or prodrug thereof, wherein:
Y is —C(O)—, —CH₂, —CH₂C(O)— or SO₂;

### X is H;

Z is (C₀₋₄-alkyl)-C(O)R³, C₁₋₄-alkyl, (C₀₋₄-alkyl)-OH, (C₁₋₄-alkyl)-O(C₁₋₄-alkyl), (C₁₋₄-alkyl)-SO₂(C₁₋₄-alkyl), (C₀₋₄-alkyl)-SO(C₁₋₄-alkyl), (C₀₋₄-alkyl)-NH₂, (C₀₋₄-alkyl)-N(C₁₋₈alkyl)₂, (C₀₋₄-alkyl)-N(H)(OH), or CH₂NSO₂(C₁₋₄-alkyl);
R₁ and R₂ are independently C₁₋₈-alkyl, cycloalkyl, or (C₁₋₄-alkyl)cycloalkyl;
R³ is NR⁴R⁵, OH, or O—(C₁₋₈-alkyl);

### R⁴ is H;

R⁵ is —OH or —OC(O)R⁶; and
R⁶ is C₁₋₈-alkyl, amino-(C₁₋₈-alkyl), (C₁₋₈-alkyl)-(C₃₋₆-cycloalkyl), C₃₋₆-cycloalkyl, phenyl, benzyl, or aryl.

In other embodiments, provided herein is a compound of the following formula:

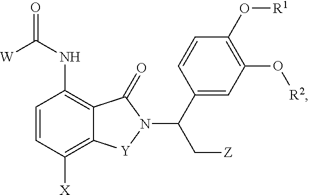

or a pharmaceutically acceptable salt, solvate, hydrate, clathrate, stereoisomer, or prodrug thereof, wherein:
Y is —C(O)—, —CH₂, —CH₂C(O)—, or SO₂;
X is halogen, —CN, —NR⁷R⁸, —NO₂, or —CF₃;
Z is (C₀₋₄alkyl)-SO₂(C₁₋₄-alkyl), —(C₀₋₄-alkyl)-CN, —(C₀₋₄-alkyl)-C(O)R³, C₁₋₄-alkyl, (C₀₋₄-alkyl)OH, (C₀₋₄-alkyl)O(C₁₋₄-alkyl), (C₀₋₄-alkyl)SO(C₁₋₄-alkyl), (C₀₋₄-alkyl)NH₂, (C₀₋₄-alkyl)N(C₁₋₈-alkyl)₂, (C₀₋₄-alkyl) N(H)(OH), (C₀₋₄-alkyl)-dichloropyridine or (C₀₋₄-alkyl)NSO₂(C₁₋₄-alkyl);
W is —C₃₋₆-cycloalkyl, —(C₁₋₈-alkyl)-(C₃₋₆-cycloalkyl), —(C₀₋₈-alkyl)-(C₃₋₆-cycloalkyl)NR₇R₈, (C₀₋₈-alkyl)-NR₇R₈, (C₀₋₄alkyl)-CHR₉—(C₀₋₄alkyl)-NR₇R₈;
R¹ and R² are independently C₁₋₈-alkyl, cycloalkyl, or (C₁₋₄-alkyl)cycloalkyl;
R³ is C₁₋₈-alkyl, NR⁴R⁵, OH, or O—(C₁₋₈-alkyl);
R⁴ and R⁵ are independently H, C₁₋₈-alkyl, (C₀₋₈-alkyl)-(C₃₋₆-cycloalkyl), OH, or —OC(O)R⁶;
R⁶ is C₁₋₈-alkyl, (C₀₋₈-alkyl)-(C₃₋₆-cycloalkyl), amino-(C₁₋₈-alkyl), phenyl, benzyl, or aryl;
R⁷ and R⁸ are each independently H, C₁₋₈-alkyl, (C₀₋₈-alkyl)-(C₃₋₆-cycloalkyl), phenyl, benzyl, or aryl; or R⁷ and R⁸ can be taken together with the atom connecting them to form a 3 to 7 membered heterocycloalkyl or heteroaryl ring;
R⁹ is C₁₋₄ alkyl, (C₀₋₄alkyl)aryl, (C₀₋₄alkyl)-(C₃₋₆-cycloalkyl), (C₀₋₄alkyl)-heterocylcle.

In one embodiment, W is

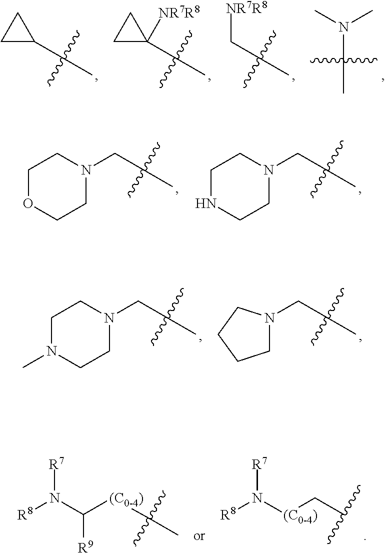

In another embodiment, representative compounds include those of formula:

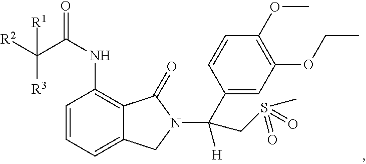

or a pharmaceutically acceptable salt, solvate, hydrate, clathrate, stereoisomer, or prodrug thereof, wherein:
R¹, R², and R³ are independently H or C₁₋₈-alkyl, with the proviso that at least one of R¹, R², and R³ is not H.

Still other PDE4 modulators include, but are not limited to, isoindoline compounds disclosed in U.S. publication no. 2006/0025457A1, published Feb. 2, 2006, which is incorporated herein by reference. Representative compounds include those listed in Table 1 below, or a pharmaceutically acceptable salt, solvate, hydrate, clathrate, stereoisomer, or prodrug thereof:

TABLE 1

| No. | Structure |
|---|---|
| 1 | 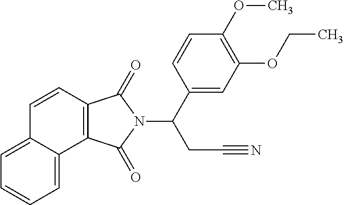 [SMILES: CCOc1cc(C(CC#N)N2C(=O)c3ccc4ccccc4c3C2=O)ccc1OC] |
| 2 | 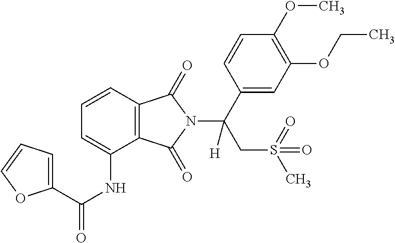 [SMILES: CCOc1cc(C(CS(C)(=O)=O)N2C(=O)c3cccc(NC(=O)c4ccco4)c3C2=O)ccc1OC] |
| 3 | 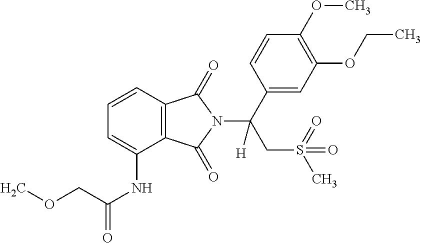 [SMILES: CCOc1cc(C(CS(C)(=O)=O)N2C(=O)c3cccc(NC(=O)COC)c3C2=O)ccc1OC] |
| 4 | 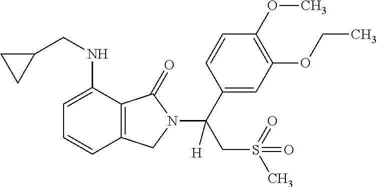 [SMILES: CCOc1cc(C(CS(C)(=O)=O)N2Cc3cccc(NCC4CC4)c3C2=O)ccc1OC] |
| 5 | 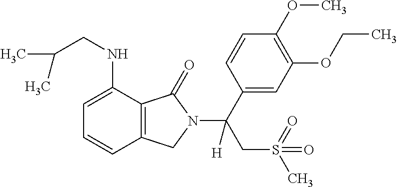 [SMILES: CCOc1cc(C(CS(C)(=O)=O)N2Cc3cccc(NCC(C)C)c3C2=O)ccc1OC] |
| 6 | 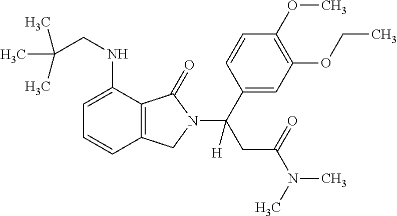 [SMILES: CCOc1cc(C(CC(=O)N(C)C)N2Cc3cccc(NCC(C)(C)C)c3C2=O)ccc1OC] |
| 7 | 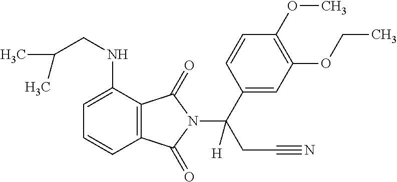 [SMILES: CCOc1cc(C(CC#N)N2C(=O)c3cccc(NCC(C)C)c3C2=O)ccc1OC] |
| 8 | 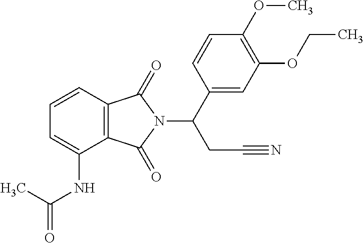 [SMILES: CCOc1cc(C(CC#N)N2C(=O)c3cccc(NC(C)=O)c3C2=O)ccc1OC] |
| 9 | 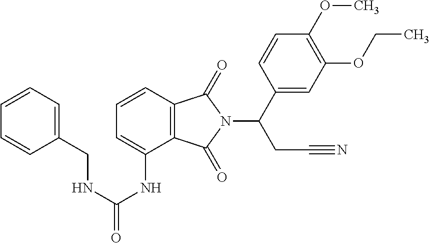 [SMILES: CCOc1cc(C(CC#N)N2C(=O)c3cccc(NC(=O)NCc4ccccc4)c3C2=O)ccc1OC] |
| 10 | 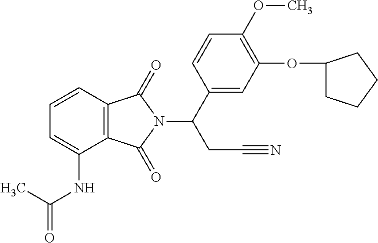 [SMILES: COc1ccc(C(CC#N)N2C(=O)c3cccc(NC(C)=O)c3C2=O)cc1OC1CCCC1] |
| 11 | 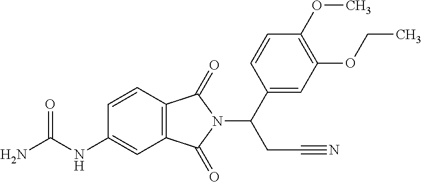 [SMILES: CCOc1cc(C(CC#N)N2C(=O)c3ccc(NC(N)=O)cc3C2=O)ccc1OC] |
| 12 | 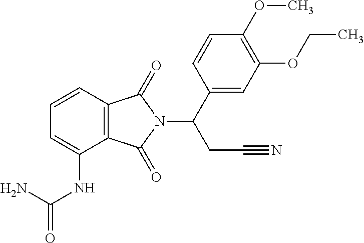 [SMILES: CCOc1cc(C(CC#N)N2C(=O)c3cccc(NC(N)=O)c3C2=O)ccc1OC] |
| 13 | 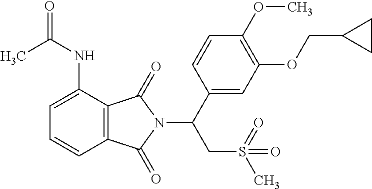 [SMILES: COc1ccc(C(CS(C)(=O)=O)N2C(=O)c3cccc(NC(C)=O)c3C2=O)cc1OCC1CC1] |
| 14 | 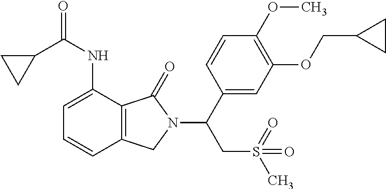 [SMILES: COc1ccc(C(CS(C)(=O)=O)N2Cc3cccc(NC(=O)C4CC4)c3C2=O)cc1OCC1CC1] |
| 15 | 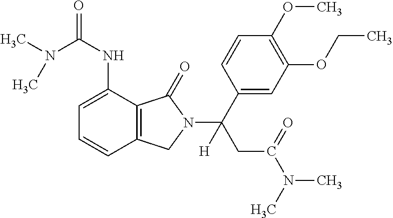 [SMILES: CCOc1cc(C(CC(=O)N(C)C)N2Cc3cccc(NC(=O)N(C)C)c3C2=O)ccc1OC] |
| 16 | 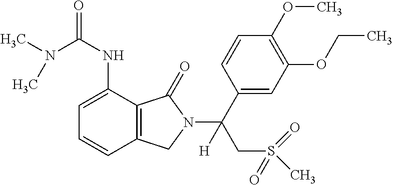 [SMILES: CCOc1cc(C(CS(C)(=O)=O)N2Cc3cccc(NC(=O)N(C)C)c3C2=O)ccc1OC] |

In another embodiment, also provided herein are 2-[1-(3-ethoxy-4-methoxyphenyl)-2-methylsulfonylethyl]-4,5-dinitroisoindoline-1,3-dione, or a pharmaceutically acceptable salt, solvate, hydrate, clathrate, stereoisomer, or prodrug thereof. In one embodiment, provided herein is a hydrochloride salt of 2-[1-(3-ethoxy-4-methoxyphenyl)-2-methylsulfonylethyl]-4,5-dinitroisoindoline-1,3-dione.

Still other PDE4 modulators include, but are not limited to, isoindoline compounds disclosed in U.S. Pat. No. 7,244,259, which is incorporated herein by reference. Representative compounds include cyclopropanecarboxylic acid {2-[1-(3-ethoxy-4-methoxy-phenyl)-2-[1,3,4]oxadiazol-2-yl-ethyl]-3-oxo-2,3-dihydro-1H-isoindol-4-yl}-amide, which has the following chemical structure, or a pharmaceutically acceptable salt, solvate, hydrate, clathrate, stereoisomer, or prodrug thereof:

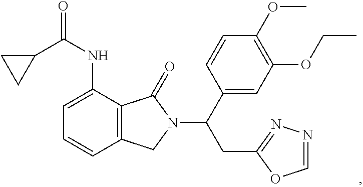 [SMILES: CCOc1cc(C(Cc2nnco2)N2Cc3cccc(NC(=O)C4CC4)c3C2=O)ccc1OC]

Still other PDE4 modulators include, but are not limited to, N-alkyl-hydroxamic acid-isoindolyl compounds disclosed in U.S. Pat. No. 6,911,464, which is incorporated herein by reference. Representative compounds include those of formula:

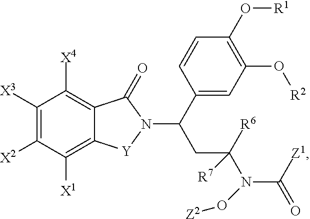

or a pharmaceutically acceptable salt, solvate, hydrate, clathrate, stereoisomer, or prodrug thereof, wherein:
Y is —C(O)—, —CH₂, —CH₂C(O)— or SO₂;
R¹ and R² are independently C₁₋₈-alkyl, CF₂H, CF₃, CH₂CHF₂, cycloalkyl, or (C₁₋₈-alkyl)cycloalkyl;
Z¹ is H, C₁₋₆-alkyl, —NH₂—NR³R⁴ or OR⁵;
Z² is H or C(O)R⁵;
X¹, X², X³ and X⁴ are each independently H, halogen, NO₂, OR₃, CF₃, C₁₋₆-alkyl, (C₀₋₄alkyl)-(C₃₋₆-cycloalkyl), (C₀₋₄-alkyl)-N—(R⁸R⁹), (C₀₋₄-alkyl)-NHC(O)—(R⁸), (C₀₋₄-alkyl)-NHC(O)CH(R⁸)(R⁹), (C₀₋₄-alkyl)-NHC(O)N(R⁸R⁹), (C₀₋₄-alkyl)-NHC(O)0(R⁸), (C₀₋₄-alkyl)-O—R₈, (C₀₋₄-alkyl)-imidazolyl, (C₀₋₄-alkyl)-pyrrolyl, (C₀₋₄-alkyl)oxadiazolyl, (C₀₋₄-alkyl)-triazolyl or (C₀₋₄-alkyl)-heterocycle;
R³, R⁴, and R⁵ are each independently H, C₁₋₆-alkyl, O—C₁₋₆-alkyl, phenyl, benzyl, or aryl;
R⁶ and R⁷ are independently H or C₁₋₆-alkyl; and
R⁸ and R⁹ are each independently H, C₁₋₉-alkyl, C₃₋₆-cycloalkyl, (C₁₋₆-alkyl)-(C₃₋₆cycloalkyl), (C₀₋₆-alkyl)-N(R⁴R⁵), (C₁₋₆-alkyl)-OR⁵, phenyl, benzyl, aryl, piperidinyl, piperizinyl, pyrolidinyl, morpholino, or C₃₋₇-heterocycloalkyl.

Still other PDE4 modulators include, but are not limited to, diphenylethylene compounds disclosed in U.S. Pat. No. 7,312,241, which is incorporated herein by reference. Representative compounds include those of formula:

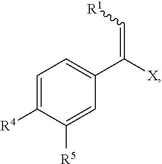

or a pharmaceutically acceptable salt, solvate, hydrate, clathrate, stereoisomer, or prodrug thereof, wherein:
R¹ is —CN, lower alkyl, —COOH, —C(O)—N(R₉)₂, —C(O)-lower alkyl, —C(O)-benzyl, —C(O)O-lower alkyl, —C(O)O-benzyl;
R⁴ is —H, —NO₂, cyano, substituted or unsubstituted lower alkyl, substituted or unsubstituted alkoxy, halogen, —OH, —C(O)(R₁₀)₂, —COOH, —NH₂, or —OC(O)—N(R₁₀)₂;
R⁵ is substituted or unsubstituted lower alkyl, substituted or unsubstituted alkoxy, or substituted or unsubstituted alkenyl;
X is substituted or unsubstituted phenyl, substituted or unsubstituted pyridine, substituted or unsubstituted pyrrolidine, substituted or unsubstituted imidizole, substituted or unsubstituted naphthalene, substituted or unsubstituted thiophene, or substituted or unsubstituted cycloalkyl; each occurrence of R⁹ is independently —H or substituted or unsubstituted lower alkyl; and each occurrence of R¹⁰ is independently —H or substituted or unsubstituted lower alkyl.

In another embodiment, representative compounds include those of formula:

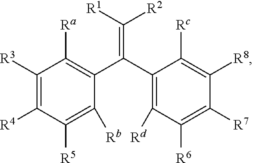

or a pharmaceutically acceptable salt, solvate, hydrate, clathrate, stereoisomer, or prodrug thereof, wherein:
R¹ and R² are independently —H, —CN, substituted or unsubstituted lower alkyl, substituted or unsubstituted alkenyl, substituted or unsubstituted alkynyl, —COOH, —C(O)-lower alkyl, —C(O)O-lower alkyl, —C(O)—N(R₉)₂, substituted or unsubstituted aryl, or substituted or unsubstituted heterocycle;
each occurrence of R^{a}, R^{b}, R^{c} and R^{d} is independently —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R³ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂; or R³ with either R^{a} or with R⁴, together form —O—C(R¹⁶R¹⁷)—O— or —O—(C(R¹⁶R¹⁷))₂—O—;
R⁴ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁵ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁶ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁷ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂;
R⁸ is —H, substituted or unsubstituted lower alkyl, substituted or unsubstituted aryl, substituted or unsubstituted heterocycle, substituted or unsubstituted cycloalkyl, substituted or unsubstituted alkoxy, halogen, cyano, —NO₂, —OH, —OPO(OH)₂, —N(R⁹)₂, —OC(O)—R¹⁰, —OC(O)—R¹⁰—N(R¹⁰)₂, —C(O)N(R¹⁰)₂, —NHC(O)—R¹⁰, —NHS(O)₂—R¹⁰, —S(O)₂—R¹⁰, —NHC(O)NH—R¹⁰, —NHC(O)N(R¹⁰)₂, —NHC(O)NHSO₂—R¹⁰, —NHC(O)—R¹⁰—N(R¹⁰)₂, —NHC(O)CH(R¹⁰)(N(R⁹)₂) or —NHC(O)—R¹⁰—NH₂, or R⁸ with either R^{c} or with R⁷, together form —O—C(R¹⁶R¹⁷)—O— or —O—(C(R¹⁶R¹⁷))₂—O—;
each occurrence of R⁹ is independently —H, substituted or unsubstituted lower alkyl, or substituted or unsubstituted cycloalkyl;
each occurrence of R¹⁰ is independently substituted or unsubstituted lower alkyl, substituted or unsubstituted cycloalkyl, substituted or unsubstituted aryl, substituted or unsubstituted lower hydroxyalkyl, or R¹⁰ and a nitrogen to which it is attached form a substituted or unsubstituted heterocycle; or R¹⁰ is —H where appropriate; and
each occurrence of R¹⁶ and R¹⁷ is independently —H or halogen.

In another embodiment, provided herein is 3-(3,4-dimethoxyphenyl)-3-(1-oxo-1,3-dihydroisoindol-2-yl)propionic acid methyl ester:

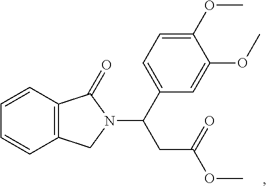 [SMILES: COC(=O)CC(c1ccc(OC)c(OC)c1)N1Cc2ccccc2C1=O]

or a pharmaceutically acceptable salt, solvate, hydrate, clathrate, stereoisomer, or prodrug thereof.

In one embodiment, provided herein are 2-[1-(3-ethoxy-4-methoxyphenyl)-2-methylsulfonylethyl]-4-acetylaminoisoindoline-1,3-dione and cyclopropyl-N-{2-[1-(3-ethoxy-4-methoxyphenyl)-2-(methylsulfonyl)ethyl]-3-oxoisoindolin-4-yl}carboxamide, which respectively have the following structures:

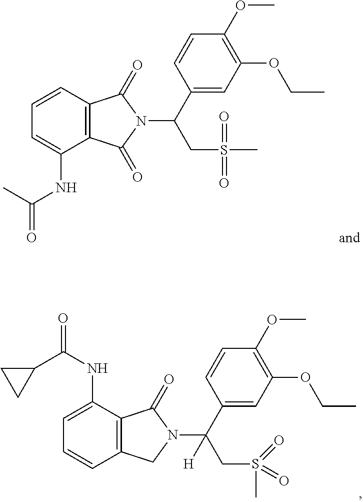 [SMILES: CCOc1cc(C(CS(C)(=O)=O)N2C(=O)c3cccc(NC(C)=O)c3C2=O)ccc1OC ; CCOc1cc(C(CS(C)(=O)=O)N2Cc3cccc(NC(=O)C4CC4)c3C2=O)ccc1OC]

or a pharmaceutically acceptable salt, solvate, hydrate, clathrate, stereoisomer, or prodrug thereof. In another embodiment, stereoisomers of these compounds are also encompassed.

Compounds provided herein can either be commercially purchased or prepared according to the methods described in the patents or patent publications disclosed herein. Further, optically pure compositions can be asymmetrically synthesized or resolved using known resolving agents or chiral columns as well as other standard synthetic organic chemistry techniques.

Various PDE4 modulators contain one or more chiral centers, and can exist as racemic mixtures of enantiomers or mixtures of diastereomers. In one embodiment, provided herein is the use of stereomerically pure forms of such compounds, as well as the use of mixtures of those forms. For example, mixtures comprising equal or unequal amounts of the enantiomers of PDE4 modulators may be used in methods and compositions provided herein. The purified (R) or (S) enantiomers of the specific compounds disclosed herein may be used substantially free of its other enantiomer.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.3 Methods of Treatment and Prevention

In certain embodiments, the invention relates to methods for treating or preventing a virus induced disease or condition ameliorated by modulating PDE4, comprising administering to a patient in need thereof an effective amount of a compound as disclosed herein, such as Compound A, Compound B, or a pharmaceutically acceptable salt, solvate, hydrate, clathrate, stereoisomer, or prodrug thereof.

In certain embodiments, the invention relates to a method for treating or preventing a virus induced disease or condition, comprising administering to a patient in need thereof an effective amount of a compound as disclosed herein, such as Compound A, Compound B, or a pharmaceutically acceptable salt, solvate, hydrate, clathrate, stereoisomer, or prodrug thereof

In certain embodiments, the invention relates to a method for treating or preventing a virus induced disease or condition associated with a hemorrhagic fever virus. In certain embodiments, hemorrhagic fever viruses include, but are not limited to, those belonging to arenaviridae, bunyaviridae, filoviridae, flaviviridae, and paramyxoviridae families of viruses. In one embodiment, the hemorrhagic fever virus belongs to the family arenaviridae. Examples of hemorrhagic fever viruses in the family arenaviridae include, but are not limited to, Lymphocytic choriomeningitis virus, Junin virus, Machupo virus, Lassa virus, Guanarito virus, Sabia virus, Chepare virus, and Lujo virus. In one embodiment, the hemorrhagic fever virus belongs to the family bunyaviridae. Examples of hemorrhagic fever viruses in the family bunyaviridae include, but are not limited to, Rift Valley fever virus and Crimean-Congo hemorrhagic fever virus. In one embodiment, the hemorrhagic fever virus belongs to the family filoviridae. Examples of hemorrhagic fever viruses in the family filoviridae include, but are not limited to, Marburgvirus and Ebolavirus. In one embodiment, the hemorrhagic fever virus belongs to the family flaviviridae. Examples of hemorrhagic fever viruses in the family flaviviridae include, but are not limited to, Yellow Fever virus, Dengue Fever virus, Japanese encephalitis virus, West Nile virus, Kyasanur Forest disease virus, Omsk hemorrhagic fever virus, and Alkhurma disease virus. In one embodiment, the hemorrhagic fever virus belongs to the family paramyxoviridae. Examples of hemorrhagic fever viruses in the family paramyxoviridae include, but are not limited to, Hendra virus and Nipah virus.

In certain embodiments, the invention relates to a method for treating or preventing a virus induced disease or condition associated with a virus selected from the family orthomyxoviridae. Examples of viruses in the orthomyxoviridae family include, but are not limited to, Influenzavirus A, Influenzavirus B, Influenzavirus C, Isavirus, Thogotovirus, and Quaranjavirus. In certain specific embodiments, the virus is Influenzavirus A, Influenzavirus B, or Influenzavirus C.

In certain embodiments, the invention relates to a method for treating or preventing a virus induced disease or condition associated with a virus selected from filovirus, arenavirus, bunyavirus, an influenza virus, a flavivirus, and any other virus that creates a cytokine storm associated with its infection. In certain such embodiments, the virus is selected from filovirus, arenavirus, bunyavirus, an influenza virus, and a flavivirus. In certain embodiments, the virus is selected from filovirus, arenavirus, bunyavirus, and a flavivirus.

In certain embodiments, the invention relates to a method for treating or preventing a virus induced disease or condition associated with an arenavirus, comprising administering to a patient in need thereof an effective amount of a compound as disclosed herein, such as Compound A, Compound B, or a pharmaceutically acceptable salt, solvate, hydrate, clathrate, stereoisomer, or prodrug thereof. In certain embodiments, the arenavirus is selected from Lymphocytic choriomeningitis virus, Lassa virus, Junin virus, Machupo virus, Guanarito virus, and Sabiá virus. In certain embodiments, the invention relates to a method for treating or preventing a virus induced disease or condition associated with Junin virus, comprising administering to a patient in need thereof an effective amount of a compound as disclosed herein, such as Compound A, Compound B or a pharmaceutically acceptable salt, solvate, hydrate, clathrate, stereoisomer, or prodrug thereof.

In certain embodiments, the invention relates to a method for treating or preventing a virus induced disease or condition associated with a flavivirus, comprising administering to a patient in need thereof an effective amount of a compound as disclosed herein, such as Compound A, Compound B or a pharmaceutically acceptable salt, solvate, hydrate, clathrate, stereoisomer, or prodrug thereof. In certain embodiments, the invention relates to a method for treating or preventing a virus induced disease or condition associated with Dengue virus.

In certain embodiments, the invention relates to a method for treating or preventing a viral hemorrhagic fever, comprising administering to a patient in need thereof an effective amount of a compound as disclosed herein, such as Compound A, Compound B, or a pharmaceutically acceptable salt, solvate, hydrate, clathrate, stereoisomer, or prodrug thereof. In certain such embodiments, the viral hemorrhagic fever is selected from Ebola hemorrhagic fever, Marburg hermorrhagic fever, Lassa fever, Argentine hemorrhagic fever, Bolivian hemorrhagic fever, Venezuelan hemorrhagic fever, Brazilian hemorrhagic fever, Crimean Congo hemorrhagic fever, hermorrhagic fever with renal syndrome, human pulmonary syndrome, and Rift valley fever.

In certain embodiments, the invention relates to a method for treating or preventing a virus induced disease or condition selected from Lymphocytic choriomeningitis, Lassa fever, Argentine hemorrhagic fever, Bolivian hemorrhagic fever, Venezuelan hemorrhagic fever, and Brazilian hemorrhagic fever, comprising administering to a patient in need thereof an effective amount of a compound as disclosed herein, such as Compound A, Compound B, or a pharmaceutically acceptable salt, solvate, hydrate, clathrate, stereoisomer, or prodrug thereof. In certain embodiments, the invention relates to a method for treating or preventing Argentine hemorrhagic fever, comprising administering to a patient in need thereof an effective amount of a compound as disclosed herein, such as Compound A, Compound B, or a pharmaceutically acceptable salt, solvate, hydrate, clathrate, stereoisomer, or prodrug thereof.

### 5.4 Combination Therapy

In certain embodiments, the invention relates to methods for treating a virus induced disease or condition, comprising administering a compound as disclosed herein, such as Compound A, Compound B, or a pharmaceutically acceptable salt, solvate, hydrate, clathrate, stereoisomer, or prodrug thereof, in combination with another medicament. Such combination therapy may be achieved by way of the simultaneous, sequential, or separate dosing of the individual components of the treatment. Additionally, when administered as a component of such combination therapy, the PDE4 modulator and the other medicament may be synergistic, such that the dose of either or both of the components may be reduced as compared to the dose of either component that would normally be given as a monotherapy. Alternatively, when administered as a component of such combination therapy, the PDE4 modulator and the other medicament may be additive, such that the dose of each of the components is similar or the same as the dose of either component that would normally be given as a monotherapy.

In certain embodiments, the other medicament is immune plasma in defined doses of specific neutralizing antibodies per kg of body weight.

In certain embodiments, the other medicament is an anti-viral agent, such as Ribavirin.

### 5.5 Pharmaceutical Compositions

Pharmaceutical compositions and single unit dosage forms of Compound A or a pharmaceutically acceptable salt, solvate, or hydrate thereof, or Compound B or a pharmaceutically acceptable salt, solvate, or hydrate thereof, are also encompassed by the invention. Individual dosage forms of the invention may be suitable for oral, mucosal (including rectal, nasal, or vaginal), parenteral (including subcutaneous, intramuscular, bolus injection, intraarterial, or intravenous), sublingual, transdermal, buccal, or topical administration.

Pharmaceutical compositions may be used in the preparation of individual, single unit dosage forms. Pharmaceutical compositions and dosage forms provided herein comprise a compound as provided herein, or a pharmaceutically acceptable salt, solvate, or hydrate thereof. Pharmaceutical compositions and dosage forms can further comprise one or more excipients.

In certain embodiments, pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose other mono- or disaccharides. As used herein, the term “lactose-free” means that the amount of lactose present, if any, is insufficient to substantially increase the degradation rate of an active ingredient.

Lactose-free compositions can comprise excipients that are well known in the art and are listed, for example, in the U.S. Pharmacopeia (USP) 25 NF20 (2002). In general, lactose-free compositions comprise active ingredients, a binder/filler, and a lubricant in pharmaceutically compatible and pharmaceutically acceptable amounts. In one embodiment, lactose-free dosage forms comprise active ingredients, microcrystalline cellulose, pre-gelatinized starch, and magnesium stearate.

Also provided are anhydrous pharmaceutical compositions and dosage forms since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

An anhydrous pharmaceutical composition should be prepared and stored such that its anhydrous nature is maintained. Accordingly, anhydrous compositions are, in one embodiment, packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

### 5.5.1 Oral Dosage Forms

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's The Science and Practice of Pharmacy, 21st Ed., Lippincott Williams & Wilkins (2005).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In one embodiment, a solid oral dosage form comprises a compound provided herein, and optional excipients, such as anhydrous lactose, microcrystalline cellulose, polyvinylpyrrolidone, stearic acid, colloidal anhydrous silica, and gelatin.

### 5.5.2 Controlled Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and U.S. Pat. Nos. 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In one embodiment, provided are single unit dosage forms suitable for oral administration such as, but not limited to, tablets, capsules, gelcaps, and caplets that are adapted for controlled-release.

In one embodiment, controlled-release pharmaceutical products improve drug therapy over that achieved by their non-controlled counterparts. In another embodiment, the use of a controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

In another embodiment, the controlled-release formulations are designed to initially release an amount of drug (active ingredient) that promptly produces the desired therapeutic or prophylactic effect, and gradually and continually release of other amounts of drug to maintain this level of therapeutic or prophylactic effect over an extended period of time. In one embodiment, in order to maintain a constant level of drug in the body, the drug can be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.5.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### 5.5.4 Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.6 Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a unit dosage form of Compound A or a pharmaceutically acceptable salt, solvate, or hydrate thereof, or Compound B or a pharmaceutically acceptable salt, solvate, or hydrate thereof, and a unit dosage form of a second active agent.

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6 EXAMPLES

### 6.1 Example 1

The pharmacokinetics of Compound A and Compound B were tested in guinea pigs. Twelve female Hartley guinea pigs that were ten to eleven weeks of age were purchased from Charles River Laboratories. The animals were assigned either to Group 1, which received treatment with Compound B at a dose of 50 mg/kg PO or Group 2, which received treatment with Compound A at 50 mg/kg PO.

After administration of Compound, blood was collected via vena cava bleed while the animals were anesthetized. All animals recovered with no issues and exhibited no abnormal symptoms due to treatment, and all animals survived to the end of the study. Temperatures for the animals stayed within normal limits and their weights increased. Results showed that the dose of Compound B needed to be increased but that the dose of Compound A was within range.

### 6.2 Example 2

The efficacy of Compound A and Compound B was tested against Junin virus infection in guinea pigs. Thirty six female Hartley guinea pigs that were ten to eleven weeks of age were purchased from Charles River Laboratories. The animals were assigned to Groups 1 to 6 as follows:

| | Virus Challenge | | |
|---|---|---|---|
| Group | Dose (PFU/100 μL) | Compound | Dose (mg/kg) |
| 1 | 1 × 10³ | B | 100 |
| 2 | 1 × 10³ | A | 50 |
| 3 | 0 | B | 100 |
| 4 | 0 | A | 50 |
| 5 | 1 × 10³ | Ribavirin | 60 |
| 6 | 1 × 10³ | PBS | — |

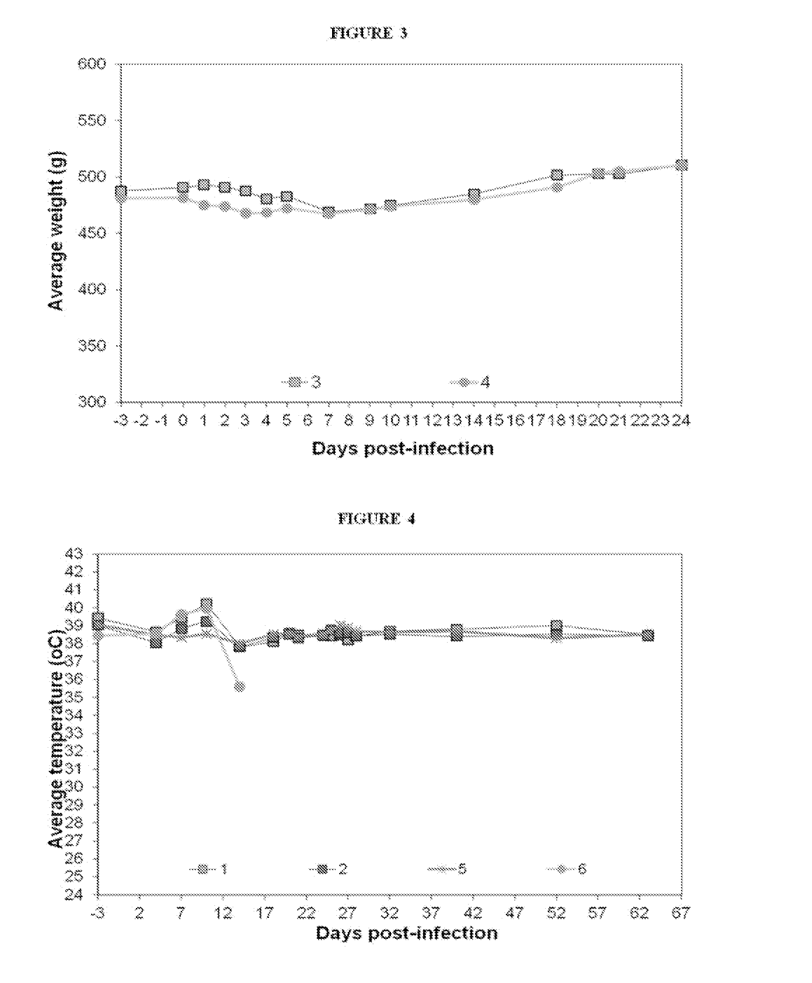

All animals in groups 3 and 4 that received Compound only with no infection showed no abnormal symptoms due to treatment and they all survived through day +28 (FIG. 3). Temperature for these animals stayed within normal limits and their weights showed an initial minor decrease, most likely from stress due to handling. Their weights started to increase after day +7.

All animals in Group 1 received Compound B and only one animal survived to the end of the study with a 17% survival rate for this group (6 animals total for survival, 3 for scheduled euthanization). Of these animals, three were found dead and two were found paralyzed and were euthanized. Death occurred starting at day +13 through day +19 post infection. Most of the animals in this group were febrile starting at day +7 but none were recorded as hypothermic. The group showed an average weight loss of 4.4%. The animal that survived showed no weight loss and never became febrile or hypothermic.

Animals in Group 2 received Compound A and 3 animals survived to the end of study with a 50% survival rate. All three animals that died were found dead between day +15 and +16 post-infection. All animals that died were febrile between day +7 and +10 and two were hypothermic on day +14. The average weight loss for this group was 0.69%. The surviving animals never became hypothermic or febrile and only showed minor weight loss between day +4 and +10.

Group 5 received Ribavirin (60 mg/kg) and all 3 animals survived to the end of study. Only 1 animal was febrile on day +27. Weigh loss up to 5.2% was seen on day +10 but animals began to gain weight after that.

Group 6 were treated with PBS and all 3 animals died or were euthanized between +14 and +17. All animals were febrile between days +7 and +10 and 2 animals were seen to be hypothermic on day +14. The average recorded weigh loss was 5.3% on day +10.

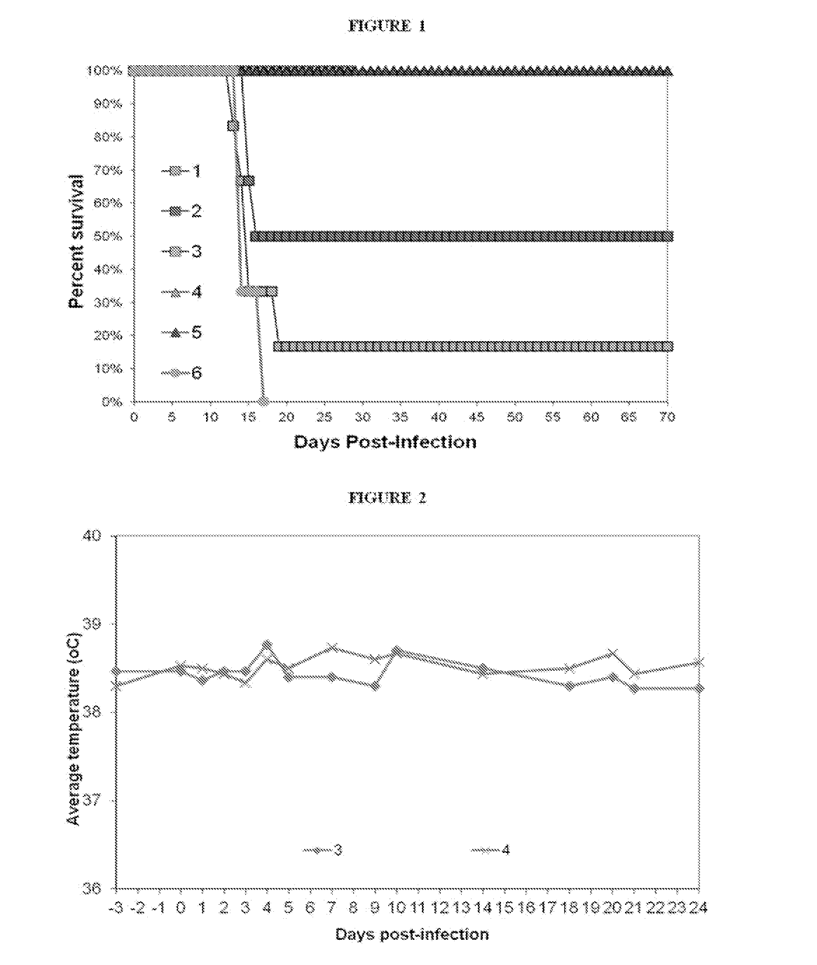

Survival, temperature and weight graphs can be seen in FIGS. 1 to 5). Scheduled euthanasia of 3 animals for group 1 and 2 was performed on day +11. The titration of the liver for Group 1 showed an average titer of 7.03×104 PFU/g and for Group 2 it was 3.67×103 PFU/g. The titration for the spleen for group 1 was 2.33×106 PFU/g and for group 2 it was 6.67×102 PFU/g. The titration of the serum for Group 1 was 1.27×105 PFU/mL and for Group 2 it was 6.67×102 PFU/mL.

It was concluded from Examples 1 and 2 that Compound A is a potential therapeutic agent for Junin infection which should be further evaluated.

### 6.3 Example 3

The efficacy of Compound A and Compound B were tested against rJunin virus infection in guinea pigs. Animals were assigned to Groups 1 to 5 as follows:

| | Virus Challenge | | |
|---|---|---|---|
| Group | Dose (PFU/100 μL) | Compound | Dose (mg/kg) |
| 1 | 1 × 10² | B | 200, PO, SID |
| 2 | 1 × 10² | A | 50, PO, SID |
| 3 | 1 × 10² | A | 50, PO, BID |
| 4 | 1 × 10² | Ribavirin | 60, IP, SID |
| 5 | 1 × 10² | PBS | 0.3 mL PO, SID |

Animals were challenged intraperitoneally with 1×10² PFU of rJunin (rRomero) virus in 100 μL of sterile PBS total volume on days 0 to 14. Compounds A and B were administered once (SID) or twice daily (BID) for fifteen days by oral route. Treatment was started 1.5-3 hours after infection on day 0 and ended on day 14. Ribavarin was administered by intra-peritoneal route once daily (SID) starting 1.5-3 hours after infection on day 0 and continuing through day +14. Total volume of test article, mock treatment, or ribavirin that were administered by oral/IP route were about 0.3-0.5 mL.

Animals in Group 1 received Compound B at a concentration of 200 mg/kg once per day. Only one animal survived to the end of study with an 11% survival rate for this group. Of these animals, three were found dead and five were euthanized. Death occurred starting day +13 through day +15 post infection. All of the animals in this group except the survivor were febrile starting day +7 thru +11 and four were recorded as hypothermic before death. The group showed an average weight loss of 12.41% on day +13. The animal that survived showed no weight loss and never became febrile or hypothermic. Blood was collected from this animal at end of study but PRNT has not yet been performed.

Animals in Group 2 received Compound A at a concentration of 50 mg/kg once per day. Three animals survived to the end of study with a 33% survival rate. One animal was found dead and the remaining five animals were euthanized, and death occurred between days +12 thru +18. All animals that died were febrile between day +7 and +15 and four out of six were hypothermic before death. The highest average weight loss for this group was 1.33% on day +14. The surviving animals never became hypothermic or febrile and showed no significant weight loss.

Animals in Group 3 received Compound A at a concentration of 50 mg/kg twice per day. Two animals survived to the end of study with a 22% survival rate. Four animals were found dead and the remaining three animals were euthanized, and death occurred between days +13 thru +16. All animals that died were febrile between day +5 and +13 and three out of seven were hypothermic before death. The highest average weight loss for this group was 5.89% on day +13. The surviving animals never became hypothermic or febrile and showed no significant weight loss.

Group 4 received Ribavirin at a concentration of 60 mg/kg once per day, and all 3 animals survived to the end of study. Only one animal was intermittently febrile between days +16 and +27. Weigh loss up to 3.09% was seen on day +13. The animal that was intermittently febrile did exhibit late symptoms of disease and had a weight loss of 17.67% by day +29. This animal would have been euthanized due to disease if the study had not ended on day +29.

Group 5 were treated with PBS and two animals died or were euthanized on day +14. The two animals that didn't survive were febrile between days +7 and +11, and one animal was seen to be hypothermic before death. The average highest recorded weigh loss was 4.38% on day +13. The surviving animal never became hypothermic or febrile and showed no significant weight loss.

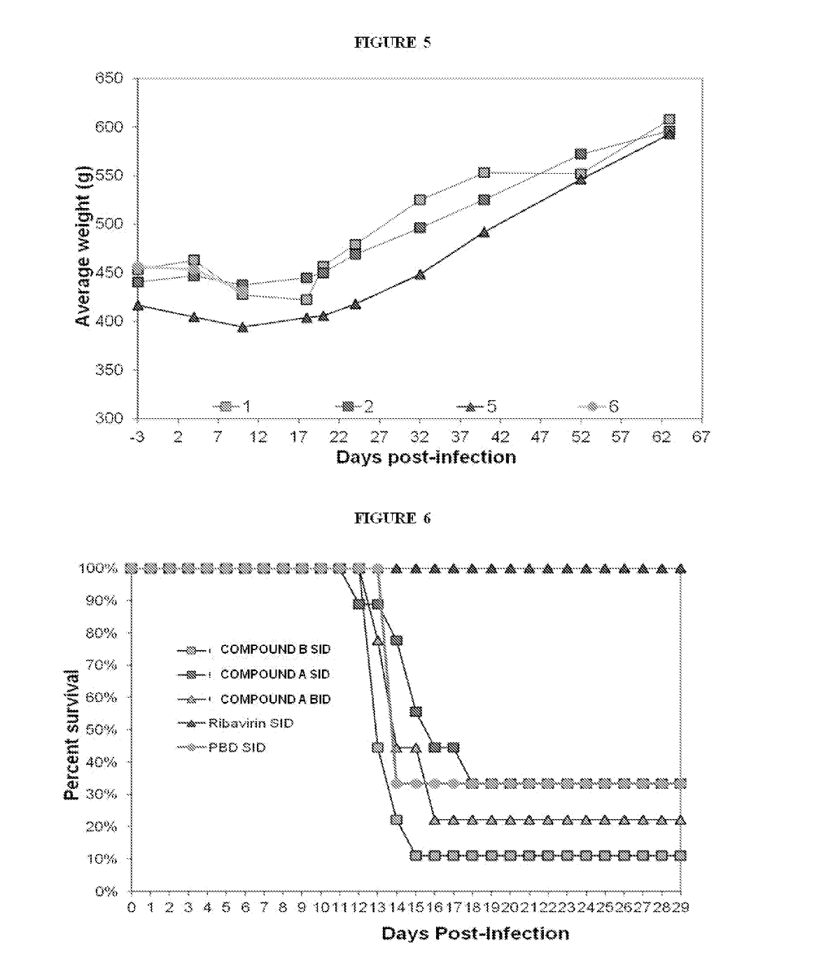

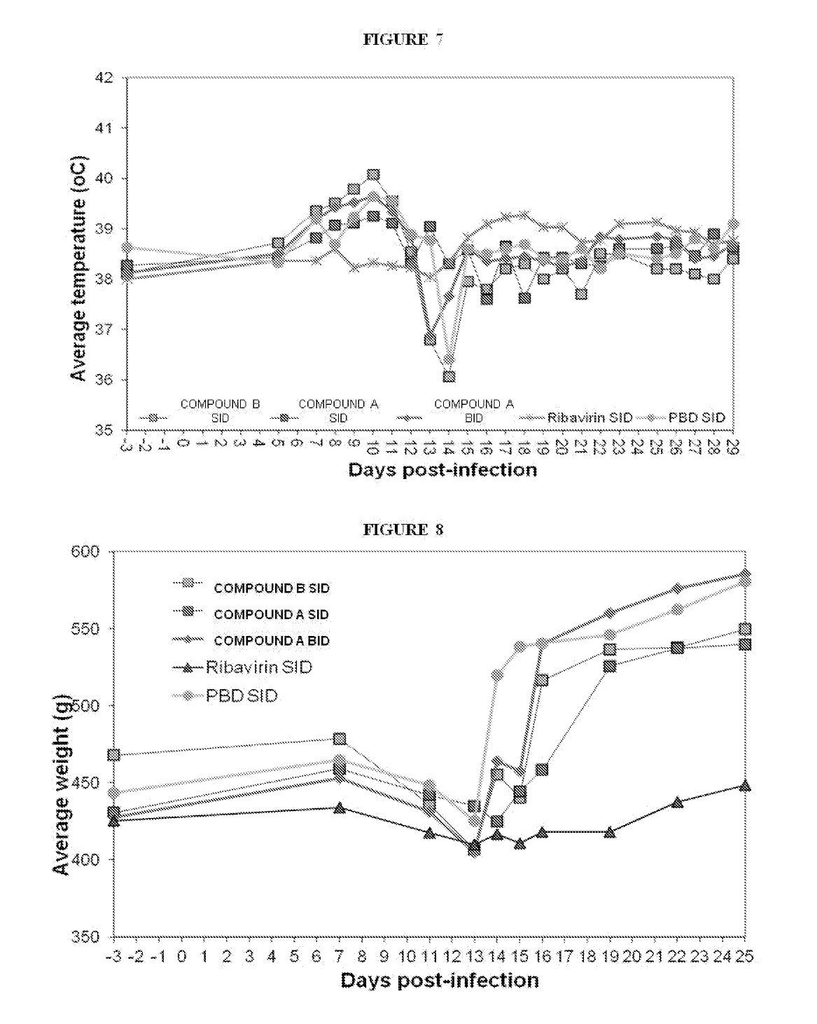

Survival, temperature and weight graphs are shown in FIGS. 6 to 8). All surviving animals were bled at the end of study to test for neutralizing antibodies against Junin virus. Hematology and clinical chemistry was also run on all surviving animals at the end of study.

It was concluded from this study that Compound A, when delivered once per day, shows promise as a potential therapeutic for Junin infection and could be used as a co-treatment drug to boost the therapeutic effect of Ribavirin. A further study using this drug as a co-treatment drug with Ribavirin is suggested.

### 6.4 Example 4

The efficacy of Compound A and Compound B were tested against Lassa (Josiah) virus infection in guinea pigs. 33 female Hartley guinea pigs that were 8-weeks of age were grouped as outlined in below. Animals were transferred to the ABSL-4 and were treated and infected on day 0. All animals were treated once daily for 15 days starting on day 0. Temperatures and weights were collected throughout the study. Surviving animals were kept through day +29, and were bled before humane euthanization. One animal was removed from study because it didn't die from Lassa virus infection, but instead was euthanized due to complications from a prolapsed anus.

| | No. of | Virus Challenge | | Antiviral dose |
|---|---|---|---|---|
| Group | Animals | Dose (PFU/100 μl) | Compound | (mg/kg) |
| 1 | 9 | 1 × 10⁴ | B | 200, PO, SID |
| 2 | 9 | 1 × 10⁴ | A | 50, PO, SID |
| 3 | 9 | 1 × 10⁴ | A | 50, PO, BID |
| 4 | 3 | 1 × 10⁴ | Ribavirin | 60, IP, SID |
| 5 | 3 | 1 × 10⁴ | PBS | 0.3 mL PO, SID |

Animals in Group 1 received Compound B at a concentration of 200 mg/kg once per day. Only 1 animal survived to the end of study with a 13% survival rate for this group. This group had the animal that was removed from study, so only eight animals are in this group. Of these animals, one was found dead and six were euthanized. Death occurred starting day +15 through day +18 post infection. All of the animals in this group except the survivor were febrile starting day +8 thru +16 and only one was recorded as hypothermic before death. The group showed an average weight loss of 13.08% on day +14. The animal that survived showed no weight loss and never became febrile or hypothermic.

Animals in Group 2 received Compound A at a concentration of 50 mg/kg once per day. None of the animals survived in this group. One animal was found dead and the remaining seven animals were euthanized, and death occurred between days +13 thru +21. All animals were febrile between day +7 and +19 and two were hypothermic before death. The highest average weight loss for this group was 12.17% on day +15.

Animals in Group 3 received Compound A at a concentration of 50 mg/kg twice per day. Four animals survived to the end of study with a 44% survival rate for this group. One animal was found dead and four animals were euthanized, and death occurred between days +13 thru +17. All animals, including the survivors, were febrile between day +7 and +17 and none of the animals were hypothermic before death. One animal was transiently hypothermic at the beginning of study between day +4 and +5. The highest average weight loss for this group was 5.36% on day +16. The surviving animals did become febrile and many showed some weight loss, especially later in the study after drug administration had ceased. On day +1 the second dose of the Compound A could not be administered.

Group 4 received Ribavirin at a concentration of 60 mg/kg once per day, and only one animal survived to the end of study. Both animals were euthanized due to disease. Only one of the two animals that died was febrile once on day +17 after it stopped receiving drug. The surviving animal was also febrile starting day +19 thru +24. Both animals that were euthanized were hypothermic, one for three days before euthanization and the other was briefly hypothermic five days before euthanization. An average group weigh loss up to 15.39% was seen on day +19. The animal that survived to the end of study also showed transient weight loss, especially after drug administration ceased.

Group 5 were treated with PBS only and two animals euthanized between days +16 and +17. The two animals that didn't survive were febrile between days +8 and +15, and one animal was seen to be hypothermic before death. The surviving animal was briefly febrile between day +11 and +13 but was never hypothermic. The average highest recorded weigh loss was 9.04% on day +17. The surviving animal showed some minor weight loss starting day +13 but recovered quickly after.

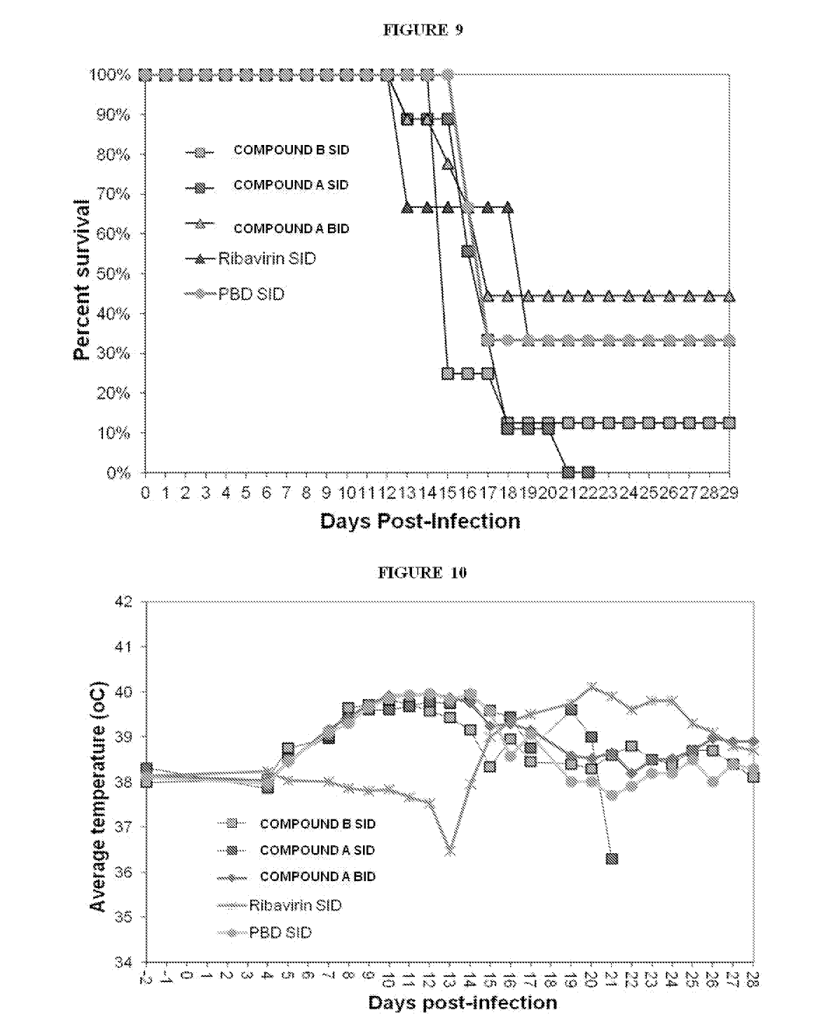

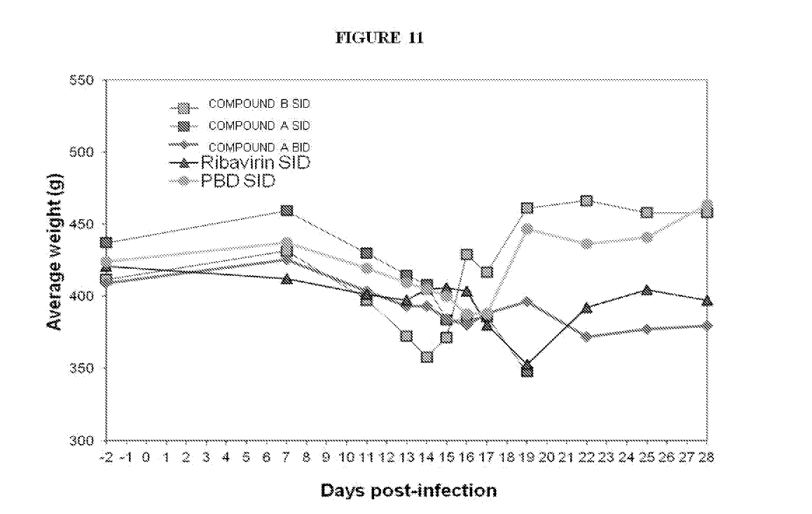

Survival, temperature and weight graphs can be seen in FIGS. 9 to 11. All surviving animals were bled at the end of study to test for neutralizing antibodies against Josiah virus. Also, hematology and clinical chemistry was also performed on all survivors.

It was concluded from this study that Compound A, when administered twice daily, shows promise as a potential therapeutic for Lassa virus infection and could possibly be used as a co-treatment drug to boost the therapeutic effect of Ribavirin. A further study using this drug as a co-treatment drug with Ribavirin is suggested.

### 6.5 Example 5

The efficacy of Compound A in combination with Ribavirin is tested against JUNV virus infection in guinea pigs. Compound A and Compound B are administered by oral route according to the following:

| | Virus Challenge | | |
|---|---|---|---|
| Group | Dose (PFU/100 μL) | Compound | Dose (mg/kg) |
| 1 | 1 × 10² | A from day 0 | 200, PO, SID |
| 2 | 1 × 10⁴ | A from day 0 and | 50, PO, SID |
| | | Ribavirin from day 8 | |
| 3 | 1 × 10⁴ | Ribavirin from day 0 | 50, PO, BID |
| 4 | 1 × 10⁴ | Ribavirin from day 8 | 60, IP, SID |
| 5 | 1 × 10⁴ | PBS | 0.3 mL PO, SID |

Animals are challenged intraperitoneally with 1×10² PFU of Junin virus in 100 μL of sterile PBS total volume. Compound A and Ribavirin are administered once (SID) daily for 15 days (Group 1 and 2 for 21 days) by oral route for Compound A and i.p for Ribavirin as described above. Treatment starts 1.5 to 3 hours after infection on day 0 and ends on day +14. Ribavirin is administered by intra-peritoneal route once daily saterting 1.5 to 3 hours after infection on day 0 and continuing through day +14. Total volume of test article, mock treatment or Ribavirin is administered by oral/IP rough is 0.3-0.5 mL.

What is claimed is:

1. A method for treating or preventing a virus induced disease or condition, comprising administering to a patient in need thereof an effective amount of 3-(3,4-dimethoxyphenyl)-3-(1-oxoindolin-2-yl)propionamide (Compound A) or a pharmaceutically acceptable salt, solvate, or hydrate thereof.

2. The method of claim 1, wherein the virus induced disease or condition is ameliorated by inhibiting PDE4 activity.

3. The method of claim 1 or 2 wherein the virus induced disease or condition is associated with a virus selected from Filovirus, arenavirus, flavivirus, orthomyxoviridae, bunyavirus, influenza virus, and any other virus that creates a cytokine storm associated with its infection.

4. The method of claim 1 or 2, wherein the virus induced disease or condition is associated with an arenavirus.

5. The method of claim 4, wherein the arenavirus is selected from Lymphocytic choriomeningitis virus, Lassa virus, Junin virus, Machupo virus, Guanarito virus, and Sabiá virus.

6. The method of claim 5, wherein the arenavirus is Junin virus.

7. The method of claim 4, wherein the virus induced disease or condition is selected from Lymphocytic choriomeningitis, Lassa fever, Argentine hemorrhagic fever, Bolivian hemorrhagic fever, Venezuelan hemorrhagic fever, and Brazilian hemorrhagic fever.

8. The method of claim 4, wherein the virus induced disease or condition is Argentine hemorrhagic fever.

9. The method of claim 4, wherein the virus induced disease or condition is Lassa fever.

10. The method of claim 1 or 2, wherein the virus induced disease or condition is a viral hemorrhagic fever.

11. The method of claim 10, wherein the viral hemorrhagic fever is selected from Ebola hemorrhagic fever, Marburg hermorrhagic fever, Lassa fever, Argentine hemorrhagic fever, Bolivian hemorrhagic fever, Venezuelan hemorrhagic fever, Brazilian hemorrhagic fever, Crimean Congo hemorrhagic fever, hermorrhagic fever with renal syndrome, human pulmonary syndrome, and Rift valley fever.

12. The method of claim 11, wherein the viral hemorrhagic fever is Rift valley fever.

13. The method of any one of claims 1 to 12, wherein Compound A is administered in combination with an anti-viral agent.

14. The method of claim 13, wherein the anti-viral agent is Ribavirin.

15. A method for treating or preventing a virus induced disease or condition, comprising administering to a patient in need thereof an effective amount of N-(2-(1-(3-ethoxy-4-methoxyphenyl)-2-(methylsulfonyl)ethyl)-3-oxoisoindolin-4-yl)cyclopropanecarboxamide (Compound B) or a pharmaceutically acceptable salt, solvate, or hydrate thereof.

16. The method of claim 15, wherein the virus induced disease or condition is ameliorated by inhibiting PDE4 activity.

17. The method of claim 15 or 16 wherein the virus induced disease or condition is associated with a virus selected from filovirus, arenavirus, bunyavirus, influenza virus, and any other virus that creates a cytokine storm associated with its infection.

18. The method of claim 15 or 16, wherein the virus induced disease or condition is associated with an arenavirus.

19. The method of claim 18, wherein the arenavirus is selected from Lymphocytic choriomeningitis virus, Lassa virus, Junin virus, Machupo virus, Guanarito virus, and Sabiá virus.

20. The method of claim 19, wherein the arenavirus is Junin virus.

21. The method of claim 20, wherein the virus induced disease or condition is selected from Lymphocytic choriomeningitis, Lassa fever, Argentine hemorrhagic fever, Bolivian hemorrhagic fever, Venezuelan hemorrhagic fever, and Brazilian hemorrhagic fever.

22. The method of claim 18, wherein the virus induced disease or condition is Argentine hemorrhagic fever.

23. The method of claim 18, wherein the virus induced disease or condition is Lassa fever.

24. The method of claim 15 or 16, wherein the virus induced disease or condition is a viral hemorrhagic fever.

25. The method of claim 24, wherein the viral hemorrhagic fever is selected from Ebola hemorrhagic fever, Marburg hermorrhagic fever, Lassa fever, Argentine hemorrhagic fever, Bolivian hemorrhagic fever, Venezuelan hemorrhagic fever, Brazilian hemorrhagic fever, Crimean Congo hemorrhagic fever, hermorrhagic fever with renal syndrome, human pulmonary syndrome, and Rift valley fever.

26. The method of claim 25, wherein the viral hemorrhagic fever is Rift valley fever.

27. The method of any one of claims 15 to 26, wherein Compound A is administered in combination with an anti-viral agent.

28. The method of claim 27, wherein the anti-viral agent is Ribavirin.

29. 3-(3,4-dimethoxyphenyl)-3-(1-oxoindolin-2-yl)propionamide (Compound A) or a pharmaceutically acceptable salt, solvate, or hydrate thereof for use in a method of treating or preventing a virus induced disease or condition.

30. Compound for use of claim 29, wherein the virus induced disease or condition is ameliorated by inhibiting PDE4 activity.

31. Compound for use of claim 29 or 30 wherein the virus induced disease or condition is associated with a virus selected from filovirus, arenavirus, bunyavirus, influenza virus, and any other virus that creates a cytokine storm associated with its infection.

32. Compound for use of claim 29 or 30, wherein the virus induced disease or condition is associated with an arenavirus.

33. Compound for use of claim 32, wherein the arenavirus is selected from Lymphocytic choriomeningitis virus, Lassa virus, Junin virus, Machupo virus, Guanarito virus, and Sabiá virus.

34. Compound for use of claim 33, wherein the arenavirus is Junin virus.

35. Compound for use of claim 32, wherein the virus induced disease or condition is selected from Lymphocytic choriomeningitis, Lassa fever, Argentine hemorrhagic fever, Bolivian hemorrhagic fever, Venezuelan hemorrhagic fever, and Brazilian hemorrhagic fever.

36. Compound for use of claim 32, wherein the virus induced disease or condition is Argentine hemorrhagic fever.

37. Compound for use of claim 32, wherein the virus induced disease or condition is Lassa fever.

38. Compound for use of claim 29 or 30, wherein the virus induced disease or condition is a viral hemorrhagic fever.

39. Compound for use of claim 38, wherein the viral hemorrhagic fever is selected from Ebola hemorrhagic fever, Marburg hermorrhagic fever, Lassa fever, Argentine hemorrhagic fever, Bolivian hemorrhagic fever, Venezuelan hemorrhagic fever, Brazilian hemorrhagic fever, Crimean Congo hemorrhagic fever, hermorrhagic fever with renal syndrome, human pulmonary syndrome, and Rift valley fever.

40. Compound for use of claim 36, wherein the viral hemorrhagic fever is Rift valley fever.

41. Compound for use of any one of claims 29 to 40, wherein Compound A is administered in combination with an anti-viral agent.

42. Compound for use of claim 41, wherein the anti-viral agent is Ribavirin.

43. N-(2-(1-(3-ethoxy-4-methoxyphenyl)-2-(methylsulfonyl)ethyl)-3-oxoisoindolin-4-yl)cyclopropanecarboxamide (Compound B) or a pharmaceutically acceptable salt, solvate, or hydrate thereof for use in a method of treating or preventing a virus induced disease or condition.

44. Compound for use of claim 43, wherein the virus induced disease or condition is ameliorated by inhibiting PDE4 activity.

45. Compound for use of claim 43 or 44 wherein the virus induced disease or condition is associated with a virus selected from filovirus, arenavirus, bunyavirus, influenza virus, and any other virus that creates a cytokine storm associated with its infection.

46. Compound for use of claim 43 or 44, wherein the virus induced disease or condition is associated with an arenavirus.

47. Compound for use of claim 46, wherein the arenavirus is selected from Lymphocytic choriomeningitis virus, Lassa virus, Junin virus, Machupo virus, Guanarito virus, and Sabiá virus.

48. Compound for use of claim 47, wherein the arenavirus is Junin virus.

49. Compound for use of claim 46, wherein the virus induced disease or condition is selected from Lymphocytic choriomeningitis, Lassa fever, Argentine hemorrhagic fever, Bolivian hemorrhagic fever, Venezuelan hemorrhagic fever, and Brazilian hemorrhagic fever.

50. Compound for use of claim 46, wherein the virus induced disease or condition is Argentine hemorrhagic fever.

51. Compound for use of claim 46, wherein the virus induced disease or condition is Lassa fever.

52. Compound for use of claim 43 or 44, wherein the virus induced disease or condition is a viral hemorrhagic fever.

53. Compound for use of claim 52, wherein the viral hemorrhagic fever is selected from Ebola hemorrhagic fever, Marburg hermorrhagic fever, Lassa fever, Argentine hemorrhagic fever, Bolivian hemorrhagic fever, Venezuelan hemorrhagic fever, Brazilian hemorrhagic fever, Crimean Congo hemorrhagic fever, hermorrhagic fever with renal syndrome, human pulmonary syndrome, and Rift valley fever.

54. Compound for use of claim 53, wherein the viral hemorrhagic fever is Rift valley fever.

55. Compound for use of any one of claims 43 to 54, wherein Compound B is administered in combination with an anti-viral agent.

56. Compound for use of claim 55, wherein the anti-viral agent is Ribavirin.
